(19) United States
(12) Patent Application Publication
(10) Pub. No.: US 2026/0209797 A1
(43) Pub. Date: Jul. 23, 2026

(54) INTEGRATING AND SELF-INACTIVATING VIRAL VECTORS AND CONSTRUCTS AND USES THEREOF

(71) Applicant: NANOCELL THERAPEUTICS HOLDINGS B.V., Utrecht (NL)

(72) Inventors: Jacek LUBELSKI, Leiden (NL); Ator ASHOTI, Utrecht (NL); Martijn EVERS, Utrecht (NL); Suneel NARAYANAVARI, Utrecht (NL)

(21) Appl. No.: 19/141,945

(22) PCT Filed: Dec. 20, 2023
(86) PCT No.: PCT/EP2023/087151

§ 371 Date: Jun. 20, 2025

Related U.S. Application Data
(60) Provisional application No. 63/476,334, filed on Dec. 20, 2022.
(60) Provisional application No. 63/595,925, filed on Nov. 3, 2023.

(51) Int. Cl. C12N 15/86; C12N 9/22; C12N 15/11

(52) U.S. Cl. CPC C12N 15/86; C12N 9/22; C12N 15/111; C12N 2310/20; C12N 2750/14143; C12N 2800/90; C12N 2830/42

(57) ABSTRACT

The present disclosure relates to gene delivery systems and methods of producing and using single constructs or viral vectors that carry a transposon and a transposase gene that is functional in target cells and not functional in producer cells.

### CROSS-REFERENCE TO RELATED APPLICATIONS

This application claims priority to U.S. Provisional Applications No. 63/595,925 filed Dec. 20, 2022, and No. 63/476,334 filed Nov. 3, 2023, each of which is incorporated by reference herein in its entirety.

### SEQUENCE LISTING

The instant application contains a Sequence Listing that has been submitted electronically in XML file format and is hereby incorporated by reference in its entirety. The Sequence Listing for this application is labeled “123690-5003-SequenceListing.xml”, which was created on Dec. 19, 2023, and is 43,800 bytes in size.

### FIELD

The present disclosure relates to gene delivery systems and methods of producing and using single constructs or viral vectors carrying a transposon and a transposase gene being functional based on their cellular environment.

### BACKGROUND

Stable and safe genomic integration of DNA encoding a sequence of interest is prerequisite for successful and durable gene transfer in many applications. Especially for gene therapy in therapeutic areas where rapidly dividing cells are affected. Rapid division of cells may result in dilution of non-integrated DNA and therefore genomic integration is required to achieve more durable gene expression and resulting therapeutic effect. Moreover, genetic disorders which affect pediatric populations where gene transfer could offer therapeutic benefit may require stable integration, as aging and resulting expansion of the subject cell population can lead to a dilution of non-integrated DNA resulting in fading of the intended therapeutic effect. Currently, there are a number of genetic tools which are used to integrate DNA cargo into the DNA of mammalian cells. This includes, but is not limited to, retroviruses, lentiviruses and non-viral transposon/transposase systems (e.g., Sleeping Beauty and PiggyBac transposon/transposase systems). In particular the Sleeping Beauty system is considered to have an unbiased, close to random integration pattern which is preferred as it seems to have a lower risk related to carcinogenicity. Current practice to deliver the transposon/transposase focuses on a two-component system wherein the transposon and the transposase are delivered separately “in trans”. The “in trans” delivery of the system allows for safe integration of only the transposon and not the transposase if this is supplied in the form of mRNA or a protein. However, it requires delivery of two separate vectors to the same cell, which makes this process relatively inefficient. On the other hand, inclusion of the transposase and the transposon into the same plasmid in “cis” configuration could result in integration of both transposon and transposase therefore posing an increased risk of carcinogenicity, due to remobilization events driven by the transposase.

To date the transposon/transposase system in “cis” configuration has only been delivered to target cells by non-viral approaches. For example, US Patent Publication No. US 2021/0324407 describes a gene delivery system using a single plasmid that carries a self-inactivating transposase gene and a corresponding transposon that is delivered to target cells by electroporation, wherein the transposase after initial transposition of a transposon inactivates itself in target cells. Chakraborty and colleagues (Chakraborty, et al., Sci. Rep. 4, 7493; DOI:10.1038/srep07403 (2014)) describe a single plasmid construct delivered by a calcium phosphate particle-based method that combines the transposase and the transposition transgene element to share a single polyA sequence resulting in self-inactivation of the transposase after transposition because polyA support for proper termination of the transposase transcript is lost as it is carried away along with the transposition transgene. These approaches in comparison to viral approaches currently lack efficient DNA delivery capabilities in vivo. Therefore, there is a need to generate a single, efficient viral delivery vector carrying the complete transposon/transposase system where the transposase is functional in target cells but not functional in producer cells.

### SUMMARY

In one aspect, disclosed herein is a polynucleotide comprising (i) a transposable element comprising a pair of inverted repeats, (ii) a first nucleic acid sequence encoding a transposase, and (iii) a mammalian intron positioned the first nucleic acid sequence.

In some embodiments, a first inverted repeat in the pair of inverted repeats is positioned upstream of the first nucleic acid sequence and a second inverted repeat in the pair of inverted repeats is positioned within the first nucleic acid sequence.

In some embodiments, the second inverted repeat is positioned within the mammalian intron.

In some embodiments, the transposable element further comprises a cargo nucleic acid sequence flanked by the pair of inverted repeats.

In some embodiments, the cargo nucleic acid sequence encodes an mRNA, a tRNA, an rRNA, an siRNA, a microRNA, a regulating RNA, or a non-coding and coding RNA.

In some embodiments, the cargo nucleic acid sequence comprises a gene of interest. In some embodiments, the cargo nucleic acid sequence encodes a therapeutic agent. In some embodiments, the cargo nucleic acid sequence does not overlap with the first nucleic acid sequence or the mammalian intron. In some embodiments, the transposable element further comprises a transcriptional regulatory element operably linked to the cargo nucleic acid sequence.

In some embodiments, the transcriptional regulatory element is a promoter. In some embodiments, the transposase is a self-integrating and self-inactivating transposase. In some embodiments, the polynucleotide further comprises a pair of AAV inverted terminal repeats flanking the transposable element and the first nucleic acid sequence.

In some embodiments, the polynucleotide is within a producer cell, the intron is not spliced thereby making the self-integrating and self-inactivating transposase not functional.

In some embodiments, when the polynucleotide is within a target cell, the intron is spliced thereby making the self-integrating and self-inactivating transposase functional.

In some embodiments, when the polynucleotide is within a target cell, the transposase splices the IRs to favor transposition and integration of the cargo in a target region of interest.

In one aspect, disclosed herein is a polynucleotide comprising (i) first nucleic acid sequence comprising a nucleic acid cassette encoding a Cas nuclease, (ii) a second nucleic acid sequence comprising nucleic acid cassette of a guide RNA (gRNA), and (iii) a mammalian intron positioned the first nucleic acid sequence.

In some embodiments, the first nucleic acid sequence comprises a nucleic acid sequence of a cargo.

In some embodiments, the cargo nucleic acid sequence encodes an mRNA, a tRNA, an rRNA, an siRNA, a microRNA, a regulating RNA, or a non-coding and coding RNA.

In some embodiments, the cargo nucleic acid sequence comprises a gene of interest.

In some embodiments, the cargo nucleic acid sequence encodes a therapeutic agent.

In some embodiments, the cargo nucleic acid sequence does not overlap with the first nucleic acid sequence or the mammalian intron.

In some embodiments, when the polynucleotide is within a producer cell, the intron is not spliced thereby making the Cas nuclease not functional.

In some embodiments, when the polynucleotide is within a target cell, the Cas nuclease binds to the gRNA thereby favoring gene repair by homology independent targeted integration (HITI) and integration of the cargo.

In one aspect, disclosed herein is a polynucleotide comprising (i) a nucleic acid sequence comprising a nucleic acid cassette encoding a ARCUS nuclease, and (ii) a mammalian intron positioned the nucleic acid sequence.

In some embodiments, the nucleic acid sequence comprises a nucleic acid sequence of a cargo.

In some embodiments, the cargo nucleic acid sequence encodes an mRNA, a tRNA, an rRNA, an siRNA, a microRNA, a regulating RNA, or a non-coding and coding RNA.

In some embodiments, the cargo nucleic acid sequence comprises a gene of interest.

In some embodiments, the cargo nucleic acid sequence encodes a therapeutic agent.

In some embodiments, the cargo nucleic acid sequence does not overlap with the first nucleic acid sequence or the mammalian intron.

In some embodiments, when the polynucleotide is within a producer cell, the intron is not spliced thereby making the ARCUS nuclease not functional.

In some embodiments, when the polynucleotide is within a target cell, the ARCUS nuclease binds to a recognition motif of ARCUS nuclease thereby favoring gene repair by excision, ligation-assisted homologous recombination (LAHR) and integration of the cargo.

In some embodiments, the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

In one aspect, disclosed herein is a delivery vehicle comprising the disclosed polynucleotides.

In some embodiments, the delivery vehicle is a lipid nanoparticle (LNP), an extracellular vesicle (EV), an exosome, or a viral vector. In some embodiments, the viral vector comprises an adeno-associated virus (AAV), recombinant adeno-associated virus (rAAV), a parvovirus, a dependovirus, an adenovirus, a lentivirus, or a SV40 virus. In some embodiments, the viral vector is an AAV. In some embodiments, the AAV comprises AAV1, AAV2, AAV3, AAV4, AAV5, AAV6, AAV7, AAV8, or AAV9.

In some embodiments, the polynucleotide is flanked by a viral vector inverted terminal repeat (ITR).

In another aspect, the present disclosure provides a cell comprising the disclosed polynucleotides.

In another aspect, the present disclosure provides a cell comprising the disclosed delivery vehicle.

In yet another aspect, the present disclosure provides an engineered cell comprising the disclosed polynucleotide or the disclosed delivery vehicle.

In one aspect, disclosed herein is a method of genetically modifying a cell comprising introducing the disclosed polynucleotides into the cell.

In one aspect, disclosed herein is a method of genetically modifying a cell comprising introducing the disclosed delivery vehicle into the cell.

In one aspect, disclosed herein is a method of expressing a gene of interest in a cell comprising introducing the disclosed polynucleotide into the cell.

In one aspect, disclosed herein is a method of expressing a gene of interest in a cell comprising introducing the disclosed delivery vehicle into the cell.

In some embodiments, the cell stably expresses the gene of interest.

In one aspect, disclosed herein is a method of treating, ameliorating or inhibiting a disease, disorder or condition in a subject in need thereof, the method comprising administering to the subject the disclosed polynucleotide or the disclosed delivery vehicle.

In one aspect, disclosed herein is a method of manufacturing or producing a delivery vehicle in producer cells, wherein the delivery vehicle comprises the disclosed polynucleotide.

In one aspect, disclosed herein is a method manufacturing or producing a cell comprising a delivery vehicle comprising the disclosed polynucleotide.

In one aspect, disclosed herein is a method for integrating a cargo nucleic acid sequence into the genome of a mammalian cell, the method comprising introducing in the mammalian cell a polynucleotide comprising (i) a transposable element comprising the cargo nucleic acid sequence and a pair of inverted repeats, (ii) a first nucleic acid sequence encoding a transposase, and (iii) a mammalian intron positioned within the first nucleic acid sequence.

In some embodiments, the transposase is activated upon the splicing of the intron.

In some embodiments, the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

In some embodiments, the activated transposase splices the IR to favor transposition and integration of the gene of interest in a target region of interest.

In one aspect, disclosed herein is a method for integrating a cargo nucleic acid sequence into the genome of a mammalian cell, the method comprising introducing in the mammalian cell a polynucleotide comprising (i) a first nucleic acid sequence comprising a nucleic acid cassette encoding a Cas nuclease, (ii) a second nucleic acid sequence comprising nucleic acid cassette of a guide RNA (gRNA), and (iii) a mammalian intron positioned the first nucleic acid sequence.

In some embodiments, the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

In some embodiments, the Cas nuclease is activated upon splicing of the intron.

In some embodiments, the activated Cas nuclease binds to the gRNA thereby favoring gene repair by homology independent targeted integration (HITI) and integration of the gene of interest.

In one aspect, disclosed herein is a method for integrating a cargo nucleic acid sequence into the genome of a mammalian cell, the method comprising introducing in the mammalian cell a polynucleotide comprising (i) a nucleic acid sequence comprising a nucleic acid cassette encoding a ARCUS nuclease, and (ii) a mammalian intron positioned the nucleic acid sequence.

In some embodiments, the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

In some embodiments, the ARCUS nuclease is activated upon splicing of the intron.

In some embodiments, the activated ARCUS nuclease binds to a recognition motif of ARCUS nuclease thereby favoring gene repair by excision, ligation-assisted homologous recombination (LAHR) and integration of the gene of interest.

In one aspect, provided herein is a viral vector for delivery of nucleic acid into a cell genome comprising:
a) a first nucleic acid encoding a transposon comprising a gene of interest; and b) a second nucleic acid encoding a transposase that is functional in target cells and not functional in producer cells.

In one aspect, provided herein is a cell comprising a viral vector wherein the vector comprises: a) a first nucleic acid encoding a transposon comprising a gene of interest; and b) a second nucleic acid encoding a transposase that is functional in target cells and not functional in producer cells.

In one aspect, provided herein is a method of genetically modifying a cell comprising introducing a viral vector of the present disclosure into a cell.

In one aspect, provided herein is a method of gene transfer in vitro and in vivo using the viral vectors of the present disclosure.

In one aspect, provided herein is a method of expressing a gene of interest in a cell comprising introducing a viral vector of the present disclosure into a cell, selecting the cell expressing the gene of interest and isolating the cells expressing a gene of interest under selective pressure.

In one aspect, provided herein is a cell prepared by introducing a viral vector of the present disclosure into the cell.

In another aspect, provided herein is a method of treating, ameliorating or inhibiting a disease, disorder or condition with the viral vectors of the present disclosure. The disease, disorder or condition can be, for example, a cancer or a viral disease.

In another aspect, provided herein is a viral vector or a cell as a medicament for treating, ameliorating or inhibiting a disease, disorder or condition with the viral vectors of the present disclosure. The disease, disorder or condition can be, for example, a cancer or a viral disease.

In yet another aspect, provided herein is a method of manufacturing or producing a viral vector in producer cells wherein the vector comprises: a) a first nucleic acid encoding a transposon comprising a gene of interest; and b) a second nucleic acid encoding a transposase that is functional in target cells and not functional in producer cells.

In another aspect, provided herein is a method of manufacturing or producing a viral vector in producer cells wherein the vector comprises: a) a first nucleic acid encoding a transposon comprising a gene of interest; and b) a second nucleic acid encoding a transposase that is functional in target cells and not functional in producer cells, wherein the vector comprises an intron and wherein the producer cells are unable to splice the intron.

In another aspect, provided herein is a method of manufacturing or producing a cell comprising a viral vector, wherein the vector comprises: a) a first nucleic acid encoding a transposon comprising a gene of interest; and b) a second nucleic acid encoding a transposase that is functional in target cells and not functional in producer cells.

In another aspect, provided herein is a construct for delivery of nucleic acid into a cell genome comprising:
a) a first nucleic acid encoding a transposon comprising a gene of interest; and b) a second nucleic acid encoding a transposase that is functional in target cells and not functional in producer cells.

In another aspect, provided herein is a construct for delivery of nucleic acid into a cell genome comprising:
a) a first nucleic acid encoding a transposon comprising a gene of interest; and b) a second nucleic acid encoding a transposase that is functional in target cells and not functional in producer cells, wherein the second nucleic acid encoding a transposase further comprises an intron that splices in mammalian cells and does not splice in producer cells.

In one aspect, provided herein is a construct for delivery of nucleic acid into a cell genome comprising:
a) a first cassette comprising:
   i) an inverted repeat of a transposon;
   ii) an enhancer;
   iii) a promoter;
   iv) an intron;
   v) a target antigen coding sequence; and
   vi) a poly(A) signal; and
b) a second cassette comprising:
   i) a promoter;
   ii) a first intron;
   iii) a first amino acid coding sequence of a transposase;
   iii) a first part of a second intron;
   iv) a transposase specific inverted repeat;
   v) a second part of the second intron;
   vi) a second amino acid coding sequence of the transpose; and
   vii) a poly(A) signal
wherein the second cassette is functional in target cells and not functional in producer cells.

In one aspect, provided herein is a construct for delivery of nucleic acid into a cell genome consisting essentially of:
a) a first cassette comprising:
   i) an inverted repeat of a transposon;
   ii) an enhancer;
   iii) a promoter;
   iv) an intron;
   v) a target antigen coding sequence; and
   vi) a poly(A) signal; and
b) a second cassette comprising:
   i) a promoter;
   ii) a first intron;
   iii) a first amino acid coding sequence of a transposase;
   iii) a first part of a second intron;
   iv) a transposase specific inverted repeat;
   v) a second part of the second intron;
   vi) a second amino acid coding sequence of the transpose; and
   vii) a poly(A) signal
wherein the second cassette is functional in target cells and not functional in producer cells.

In one aspect, provided herein is a viral vector for delivery of nucleic acid into a cell genome comprising:
a) a first cassette comprising:
   i) an inverted repeat of a transposon;
   ii) an enhancer;
   iii) a promoter;
   iv) an intron;
   v) a target antigen coding sequence; and
   vi) a poly(A) signal; and
b) a second cassette comprising:
   i) a promoter;
   ii) a first intron;
   iii) a first amino acid coding sequence of a transposase;
   iii) a first part of a second intron;
   iv) a transposase specific inverted repeat;
   v) a second part of the second intron;
   vi) a second amino acid coding sequence of the transpose; and
   vii) a poly(A) signal
wherein the second cassette is functional in target cells and not functional in producer cells.

In one aspect, provided herein is a viral vector for delivery of nucleic acid into a cell genome consisting essentially of:
a) a first cassette comprising:
   i) an inverted repeat of a transposon;
   ii) an enhancer;
   iii) a promoter;
   iv) an intron;
   v) a target antigen coding sequence; and
   vi) a poly(A) signal; and
b) a second cassette comprising:
   i) a promoter;
   ii) a first intron;
   iii) a first amino acid coding sequence of a transposase;
   iii) a first part of a second intron;
   iv) a transposase specific inverted repeat;
   v) a second part of the second intron;
   vi) a second amino acid coding sequence of the transpose; and
   vii) a poly(A) signal
wherein the second cassette is functional in target cells and not functional in producer cells.

In some embodiments, the gene of interest is flanked by inverted repeats of a transposon. In certain embodiments, the nucleic acid of the transposase is disrupted with an intron. In certain embodiments, the nucleic acid of the transposase splices in target cells and is not spliced in producer cells. In certain embodiments, the nucleic acids of the transposon and transposase are flanked by inverted terminal repeats of the construct or viral vector.

These and other aspects of the viral vectors, constructs, cells and methods will be apparent upon reference to the following description. All references, patents or applications, U.S. or foreign, cited herein, which describe in more detail certain background information, procedures, methods and/or compositions are hereby incorporated by reference as if written herein in their entireties. Where any inconsistencies arise, material literally disclosed herein controls.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Schematic representation of a construct and self-integrating viral vector. ITR: inverted terminal repeats of AAV required for vector assembly and packaging. IR: inverted repeats of Sleeping Beauty required for cassette integration; SB: Sleeping Beauty, enzyme responsible for transposition; AI: artificially introduced intron, placed in the open reading frame of Sleeping Beauty, harboring IR.

FIG. 2: Schematic representation of a viral vector in mammalian cells, wherein the viral vector contains a transposon and a transposase which is functional in mammalian cells and not functional in non-mammalian production cells. 1) AAV delivers the DNA to the nucleus of a mammalian cell of interest; 2) SB mRNA is expressed, and the AI is spliced out resulting in mRNA encoding intact SB; 3) Expressed SB protein performs transposition reaction using IR's and the SB open reading frame is destroyed; and 4) Transposon is inserted into the genome.

FIGS. 3A-3B: Schematic representation of methods of making integrating viral vectors in non-mammalian and mammalian production hosts. FIG. 3A: shows that the production of rAAV-BCMA-siSB-A is possible in non-mammalian (e.g., insect cells) as it does not lead to destruction of the vector genome due to lack of splicing of intron A in the open reading frame of the Sleeping Beauty gene. FIG. 3B: shows that the production of rAAV-BCMA-siSB-A in a mammalian production host is not possible as it leads to destruction of the vector genome due to intended splicing.

FIGS. 4A-4B: Construct design. FIG. 4A: showing BCMA-CAR expression cassette, followed by Sleeping Beauty transposase expression cassette, which is disrupted by an intron (A or C variant). BCMA-CAR expression cassette is flanked by inverted repeats of transposon such that one of the IRs is also present within the intron that disrupts the Sleeping Beauty transposase open reading frame. FIG. 4B: shows the same construct design with the exception that the Sleeping Beauty transposase coding frame is scrambled by inclusion of stop codons at the beginning such that protein is not made.

FIGS. 5A-5C: Plasmid constructs harboring BCMA-CAR-siSB or BCMA-CAR-siSB-scrambled were tested for correct splicing in target cells (FIG. 5A). Detection of Sleeping Beauty transposase expression by western blotting wherein Sleeping Beauty transposase expression was detected only in cells transfected with siSB intron-A construct but not in cells transfected with siSB intron-A-scrambled control (FIG. 5B). Stable integration of the BCMA-CAR (gene of interest) into target cells genome leading to stable expression of BCMA-CAR overtime (FIG. 5C).

FIG. 6: Lack of splicing in insect cells. Constructs are not spliced in insect cells, which will allow for generation of viral vectors. Two plasmids BCMA-CAR-siSB and BCMA-CAR-siSB-scrambled were electroporated into insect cells. Sleeping Beauty mRNA product was investigated by two sperate PCR targets, as evidenced by the banding pattern, the detected fragments show lack of splicing of produced mRNA.

FIGS. 7A-7B: Production and characterization of rAAV in HEK cells. Two rAAV vectors BCMA-CAR-siSB and BCMA-CAR-siSB-scrambled were used to transduce HEK cells. FIG. 7A: Both vectors show spliced (functional transposase) and unspliced (non-functional transposase) products of Sleeping Beauty transposase mRNA product as investigated by two sperate PCR targets. FIG. 7B: BCMA-CAR-siSB and BCMA-CAR-siSB-scrambled controlled shows expression of BCMA in HEK cells. FIG. 7C: Detection of Sleeping Beauty transposase expression by western blotting wherein Sleeping Beauty transposase expression was detected only in cells transduced with rAAV expressing siSB intron-A construct but not in cells transduced with rAAV expressing siSB intron-A-scrambled control.

FIGS. 8A-8C: Schematic representation of the rAAV-siCAS mechanism when (FIG. 8A) introducing the Cas target sequence only in the Cas CDS, and when (FIG. 8B) also introducing the same Cas target sequence upstream of the GOI cassette. (FIG. 8C) Figure legend.

FIGS. 9A-9C: Schematic representation of the rAAV-siARCUS mechanism when (FIG. 9A) introducing the ARCUS target sequence only in the ARCUS CDS, and when (FIG. 9B) also introducing the same ARCUS target sequence upstream of the GOI cassette. (FIG. 9C) Figure legend.

The disclosed nucleic acid sequences of the interest (SEQ ID NOs: 1-14) are provided elsewhere herein in the Examples section.

### DETAILED DESCRIPTION

This description is intended only to acquaint others skilled in the art with the present disclosure, its principles, and its practical application so that others skilled in the art may adapt and apply the disclosure in its numerous forms, as they may be best suited to the requirements of a particular use. This description and its specific examples are intended for purposes of illustration only. This disclosure, therefore, is not limited to the embodiments described in this patent application and may be variously modified.

### Definitions

In the following description, certain specific details are set forth to provide a thorough understanding of various embodiments. However, one skilled in the art will understand that the disclosure may be practiced without these details. In other instances, well-known structures and elements have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” or “some embodiments” or “a certain embodiment” or “certain embodiments” and the like means that a particular feature, structure, element or characteristic described in connection with the embodiment(s) is included in at least one embodiment. Thus, the appearances of the phrases “in one embodiment” or “in an embodiment” or “in some embodiments” or “in a certain embodiment” or “in certain embodiments” and the like in various places throughout this specification and claims are not necessarily all referring to the same embodiment. Furthermore, the particular features, structures, elements or characteristics may be combined in any suitable manner in one or more embodiments.

Also, as used in this specification and the appended claims, the singular forms “a,” “an,” and “the” include plural referents unless the content clearly dictates otherwise.

When ranges of values are disclosed, and the notation “of at least n₁ . . . or n₂” or “between n₁ . . . and n₂” is used, where n₁ and n₂ are the numbers, then unless otherwise specified, this notation is intended to include the numbers themselves and the range between them.

As used herein:

“Functional nucleic acid” has its plain and ordinary meaning when read in light of the specification, and may include, but is not limited to a nucleic acid capable of expression of a protein that can perform its function in an appropriate cell type (e.g., target cells).

“Not functional nucleic acid,” “non-functional nucleic acid” and the like have their plain and ordinary meanings when read in light of the specification, and may include, but are not limited to a nucleic acid capable of expression of a part of a protein or a full protein that is incapable of performing its function in a producer cell (e.g., insect cell). That is, the protein is partially or completely inactive (e.g., partially or completely inhibited expression of protein) in the producer cell (e.g., insect cell).

“Construct(s)” has its plain and ordinary meaning when read in light of the specification, and may include, but is not limited to, a non-viral polynucleotide construct, e.g., a plasmid, cosmid or phage, used to transmit genetic material to a producer cell.

A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

“Viral vector(s)” has its plain and ordinary meaning when read in light of the specification and may include, but is not limited to, a viral polynucleotide construct, e.g., a virus, used to transmit genetic material to a target cell. A viral vector is composed of either

DNA or RNA. In some embodiments, the viral vector comprises DNA. “Self-inactivating viral vector(s)” has its plain and ordinary meaning when read in light of the specification, and may include, but is not limited to a viral vector that upon transduction of target cells expresses its gene(s) that result in cleavage of the viral vector genome resulting in its inactivation.

“Transposon” has its plain and ordinary meaning when read in light of the specification, and may include, but is not limited to a DNA sequence that can be transposed within a gene.

“Transposase” has its plain and ordinary meaning when read in light of the specification, and may include, but is not limited to an enzyme that binds to the end of a transposon and catalyzes the movement of the transposon to another part of the genome by a cut-and-paste mechanism or a replicative-transposition mechanism.

“Target cell(s)” means mammalian cells (e.g., human cells) that are modified by viral vectors for expression of a gene of interest. A number of mammalian cell lines are known in the art and include immortalized cell lines available from the American Type Culture Collection (A.T.C.C.), such as, but not limited to, Chinese hamster ovary (CHO) cells, 293 cells, HeLa cells, baby hamster kidney (BHK) cells, mouse myeloma (SB20), monkey kidney cells (COS), as well as others.

“Producer cell(s),” “production cell(s),” “production host” and the like mean(s) nonmammalian cells (e.g., eucaryotic, non-mammalian cells, insect cells, bacterial cells, plant cells, or yeast cells) that are used to produce viral vectors.

“Integrating,” “integrated,” “integration” and the like has their plain and ordinary meanings when read in light of the specification, and may include, but is not limited to a segment of DNA that has been incorporated into a chromosome of a producer cell or a mammalian target cell after that element is introduced (or delivered) into the cell through human manipulation. Such integrated DNA is passed from the original host cell or target cell to its/their progeny.

“Self-integrating” has its plain and ordinary meaning when read in light of the specification, and may include, but is not limited to a vector that is capable of integrating its entire or part of its entire genetic material to a host chromosome.

The term “intron” or “intron sequence” refers to a non-coding sequence within a gene that is removed by RNA splicing during modification of the precursor messenger RNA into mature messenger RNA (mRNA). Thus, the term refers to both the DNA sequence within a gene and the corresponding sequence in the unprocessed precursor messenger RNA transcript. Where a nucleic acid sequence encoding a gene comprises nucleic acids which together with an inserted sequence form a consensus splice donor/acceptor sequence, an intron may be inserted at this position. The inserted sequences are then spliced out during post-transcriptional processing. Spliceosomal introns are found in coding genes of eukaryotes and utilize spliceosomes for splicing (Hube F. et al., Mammalian introns: when the junk generates molecular diversity. Int J Mol Sci. 2015 Feb. 20; 16(3)).

In some embodiments, a “mammalian intron” refers to an intron that is spliced out of a sequence at a significantly higher rate when in a mammalian cell than when in a nonmammalian eukaryotic producer cell. In some embodiments, a mammalian cell is spliced out of a sequence when present in a mammalian cell at a rate that is at least 3-fold faster than the rate at which the intron is spliced out of the sequence when present in a non-mammalian eukaryotic producer cell. In some embodiments, a mammalian cell is spliced out of a sequence when present in a mammalian cell at a rate that is at least 10-fold faster than the rate at which the intron is spliced out of the sequence when present in a non-mammalian eukaryotic producer cell. In some embodiments, a mammalian cell is spliced out of a sequence when present in a mammalian cell at a rate that is at least 25-fold faster than the rate at which the intron is spliced out of the sequence when present in a non-mammalian eukaryotic producer cell. In some embodiments, a mammalian cell is spliced out of a sequence when present in a mammalian cell at a rate that is at least 50-fold faster than the rate at which the intron is spliced out of the sequence when present in a non-mammalian eukaryotic producer cell. In some embodiments, a mammalian cell is spliced out of a sequence when present in a mammalian cell at a rate that is at least 100-fold faster than the rate at which the intron is spliced out of the sequence when present in a non-mammalian eukaryotic producer cell. In some embodiments, a mammalian cell is spliced out of a sequence when present in a mammalian cell at a rate that is at least 500-fold faster than the rate at which the intron is spliced out of the sequence when present in a non-mammalian eukaryotic producer cell. In some embodiments, a mammalian cell is spliced out of a sequence when present in a mammalian cell at a rate that is at least 1000-fold faster than the rate at which the intron is spliced out of the sequence when present in a nonmammalian eukaryotic producer cell. In some embodiments, a mammalian cell is spliced out of a sequence when present in a mammalian cell at a rate that is at least 10-fold faster, at least 100-fold faster, at least 1000-fold faster, at least 10,000-fold faster, at least 10\hat{ }5-fold faster, at least 10\hat{ }6-fold faster, at least 10\hat{ }7-fold faster, at least 10\hat{ }8-fold faster, or at least 10\hat{ }9 fold faster than the rate at which the intron is spliced out of the sequence when present in a nonmammalian eukaryotic producer cell.

### DESCRIPTION

The present disclosure pertain to gene delivery vehicles and methods of making and using such vehicles, using a single construct or viral vector carrying a transposon and a transposase that is functional in target cells and not functional in producer cells. Some embodiments include nucleic acids encoding such transposes and transposon, constructs and viral vectors comprising such nucleic acids, such constructs and viral vectors comprising introns, compositions comprising such nucleic acids, constructs and vectors, methods of producing such gene delivery vehicles and methods of using such gene delivery vehicles. Some embodiments include generation or production of delivery vehicles capable of DNA integration and compositions comprising such delivery vehicles.

Provided herein are compositions and methods to generate constructs and viral vectors wherein a transposon and a transposase are combined in one construct or viral vector and are functional in one environment (e.g., target cells) but not the other (e.g., producer cells).

In some embodiments, the compositions and methods disclosed herein allow for the production of a self-integrating, self-destroying gene transfer constructs in a non-mammalian cell by inserting an exon into the coding sequence of the encoded integration enzyme (e.g., a transposase, a Cas protein, or an ARCUS protein) that is excised by the splicing machinery of a target mammalian cell but not substantially excised by the plicing machinery of nonmammalian eukaryotic production cells, e.g., insect cells used in a baculovirus expression system. In contrast, conventional polynucleotides harboring self-integrating, self-destroying gene transfer constructs cannot be efficiently packaged into viral delivery vehicles (e.g., adenoassociated viruses “AAVs”) assembled in eukaryotic cells (e.g., including insect cells) because the intron used to disrupt the integration enzyme is excised during the manufacturing process, resulting in activation of the integration enzyme and destruction of the polynucleotide by the integrating enzyme. Accordingly, the compositions and methods described herein overcome this problem, at least in part, by suppressing intron splicing during production through use of intron sequences that are not substantially excised in a non-mammalian eukaryotic production cell, e.g., an insect cell.

### Delivery Vehicles

The present disclosure provides delivery vesicles for delivering polynucleotides. Such delivery vesicles or systems within the scope of the present invention may be provided in any form, including but not limited to solid, semi-solid, emulsion, or colloidal particles. As such, any of the delivery systems described herein, including but not limited to, e.g., lipid-based systems, lipid nanoparticles (LNPs), liposomes, micelles, microvesicles, extracellular vesicles, exosomes, or gene gun may be provided as particle delivery systems within the scope of the present invention.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate. It will be understood that the size of the particle will differ depending as to whether it is measured before or after loading. Accordingly, in particular embodiments, the term “nanoparticles” may apply only to the particles pre-loading. Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. [0138] Semi-solid and soft nanoparticles have been manufactured and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants.

Self-assembling export compartments or nanoparticles with RNA may be constructed with polyethyleneimine (PEI) that is PEGylated with an Arg-Gly-Asp (RGD) peptide ligand attached at the distal end of the polyethylene glycol (PEG). This system has been used, for example, as a means to target tumor neovasculature expressing integrins and deliver siRNA inhibiting vascular endothelial growth factor receptor-2 (VEGF R2) expression and thereby achieve tumor angiogenesis (see, e.g., Schiffelers et al., Nucleic Acids Research, 2004, Vol. 32, No. 19). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes. A dosage of about 100 to 200 mg of CRISPR Cas is envisioned for delivery in the self-assembling nanoparticles of Schiffelers et al.

The lipid particles developed by Qiaobing Xu's lab at Tufts University may be used/adapted to the present delivery system. See Wang et al., J. Control Release, 2017 Jan. 31. pii: SO 168-3659(17)30038-X. doi: 10.1016/j.jconrel.2017.01.037. [Epub ahead of print]; Altmoglu et al., Biomater Sci., 4(12): 1773-80, Nov. 15, 2016; Wang et al., PNAS, 113(11):2868-73 Mar. 15, 2016; Wang et al., PloS One, 10(11): e0141860. doi: 10.1371/journal. pone.0141860. eCollection 2015, Nov. 3, 2015; Takeda et al., Neural Regen Res. 10(5):689-90, May 2015; Wang et al., Adv. Healthc Mater., 3(9): 1398-403, September 2014; and Wang et al., Agnew Chem Int Ed Engl., 53(11):2893-8, Mar. 10, 2014.

US Patent Publication No. 20110293703 also provides libraries of aminoalcohol lipidoid compounds prepared by the inventive methods. These aminoalcohol lipidoid compounds may be prepared and/or screened using high-throughput techniques involving liquid handlers, robots, microtiter plates, computers, etc. In certain embodiments, the aminoalcohol lipidoid compounds are screened for their ability to transfect polynucleotides or other agents (e.g., proteins, peptides, small molecules) into the cell.

US Patent Publication No. 2013/0302401 relates to a class of poly(beta-amino alcohols) (PBAAs) that are prepared using combinatorial polymerization. The inventive PBAAs may be used in biotechnology and biomedical applications as coatings (such as coatings of films or multilayer films for medical devices or implants), additives, materials, excipients, non-biofouling agents, micropatterning agents, and cellular encapsulation agents. When used as surface coatings, these PBAAs elicited different levels of inflammation, both in vitro and in vivo, depending on their chemical structures. The large chemical diversity of this class of materials allowed identification of polymer coatings that inhibit macrophage activation in vitro. Furthermore, these coatings reduce the recruitment of inflammatory cells, and reduce fibrosis, following the subcutaneous implantation of carboxylated polystyrene microparticles. These polymers may be used to form polyelectrolyte complex capsules for cell encapsulation. The invention may also have many other biological applications such as antimicrobial coatings, DNA or siRNA delivery, and stem cell tissue engineering. The teachings of US Patent Publication No. 20130302401 may be applied to a CRISPR Cas system or any other system of the present invention.

In another embodiment, lipid nanoparticles (LNPs) are contemplated. An antitransthyretin small interfering RNA has been encapsulated in lipid nanoparticles and delivered to humans (see, e.g., Coelho et al., N Engl J Med 2013; 369:819-29), and such a system may be adapted and applied to a CRISPR Cas system or any other system of the present invention. Doses of about 0.01 to about 1 mg per kg of body weight administered intravenously are contemplated. Medications to reduce the risk of infusion-related reactions are contemplated, such as dexamethasone, acetaminophen, diphenhydramine or cetirizine, and ranitidine are contemplated. Multiple doses of about 0.3 mg per kilogram every 4 weeks for five doses are also contemplated.

Zhu et al. (US20140348900) provides for a process for preparing liposomes, lipid discs, and other lipid nanoparticles using a multi-port manifold, wherein the lipid solution stream, containing an organic solvent, is mixed with two or more streams of aqueous solution (e.g., buffer). In some aspects, at least some of the streams of the lipid and aqueous solutions are not directly opposite of each other. Thus, the process does not require dilution of the organic solvent as an additional step. In some embodiments, one of the solutions may also contain an active pharmaceutical ingredient (API). This invention provides a robust process of liposome manufacturing with different lipid formulations and different payloads. Particle size, morphology, and the manufacturing scale can be controlled by altering the port size and number of the manifold ports, and by selecting the flow rate or flow velocity of the lipid and aqueous solutions.

LNPs have been shown to be highly effective in delivering siRNAs to the liver (see, e.g., Tabernero et al., Cancer Discovery, April 2013, Vol. 3, No. 4, pages 363-470) and are therefore contemplated for delivering RNA encoding CRISPR Cas to the liver. A dosage of about four doses of 6 mg/kg of the LNP every two weeks may be contemplated. Tabernero et al. demonstrated that tumor regression was observed after the first 2 cycles of LNPs dosed at 0.7 mg/kg, and by the end of 6 cycles the patient had achieved a partial response with complete regression of the lymph node metastasis and substantial shrinkage of the liver tumors. A complete response was obtained after 40 doses in this patient, who has remained in remission and completed treatment after receiving doses over 26 months. Two patients with RCC and extrahepatic sites of disease including kidney, lung, and lymph nodes that were progressing following prior therapy with VEGF pathway inhibitors had stable disease at all sites for approximately 8 to 12 months, and a patient with PNET and liver metastases continued on the extension study for 18 months (36 doses) with stable disease.

In some embodiments, the LNP contains a nucleic acid, wherein the charge ratio of nucleic acid backbone phosphates to cationic lipid nitrogen atoms is about 1:1.5-7 or about 1:4. In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxy ethylglucose (HEG) based polymers, polyhydroxyethyl starch (polyHES) and polypropylene. In some embodiments, the PEG, HEG, polyHES, and a polypropylene weigh between about 500 to 10,000 Da or between about 2000 to 5000 Da. In some embodiments, the shielding compound is PEG2000 or PEG5000.

In some embodiments, sugar-based particles may be used, for example GalNAc, as described herein and with reference to WO2014118272 (incorporated herein by reference) and Nair, J K et al., 2014, Journal of the American Chemical Society 136 (49), 16958-16961) and the teaching herein, especially in respect of delivery applies to all particles unless otherwise apparent. This may be considered to be a sugar-based particle and further details on other particle delivery systems and/or formulations are provided herein. GalNAc can therefore be considered to be a particle in the sense of the other particles described herein, such that general uses and other considerations, for instance delivery of said particles, apply to GalNAc particles as well. A solution-phase conjugation strategy may for example be used to attach triantennary GalNAc clusters (mol. wt.—2000) activated as PFP (pentafluorophenyl) esters onto 5′-hexylamino modified oligonucleotides (5′-HA ASOs, mol. wt.—8000 Da; Gstergaard et al., Bioconjugate Chem., 2015, 26 (8), pp 1451-1455). Similarly, poly(acrylate) polymers have been described for in vivo nucleic acid delivery (see WO2013158141 incorporated herein by reference). In further alternative embodiments, pre-mixing CRISPR nanoparticles (or protein complexes) with naturally occurring serum proteins may be used in order to improve delivery (Akinc A et al, 2010, Molecular Therapy vol. 18 no. 7, 1357-1364).

Exosomes are endogenous nano-vesicles that transport RNAs and proteins, and which can deliver RNA to the brain and other target organs. To reduce immunogenicity, Alvarez-Erviti et al. (2011, Nat Biotechnol 29: 341) used self-derived dendritic cells for exosome production. Targeting to the brain was achieved by engineering the dendritic cells to express Lamp2b, an exosomal membrane protein, fused to the neuron-specific RVG peptide. Purified exosomes were loaded with exogenous RNA by electroporation. Intravenously injected RVG-targeted exosomes delivered GAPDH siRNA specifically to neurons, microglia, oligodendrocytes in the brain, resulting in a specific gene knockdown. Pre-exposure to RVG exosomes did not attenuate knockdown, and non-specific uptake in other tissues was not observed. The therapeutic potential of exosome-mediated siRNA delivery was demonstrated by the strong mRNA (60%) and protein (62%) knockdown of BACE1, a therapeutic target in Alzheimer's disease. The teachings of Alvarez-Erviti et al. can be applied and/or adapted to generate and/or deliver the CRISPR-Cas system molecules described herein.

In some embodiments, the delivery vesicle is a virus-like particle (VLP). As used herein, the term “virus-like particle” (VLP) refers to a structure that in at least one attribute resembles a virus, but which has not been demonstrated to be infectious. A VLP may be a nonreplicating, noninfectious viral shell that contains a viral capsid but lacks all or part of the viral genome, in particular, the replicative components of the viral genome. VLPs are generally composed of one or more viral proteins, such as, but not limited to those proteins referred to as capsid, coat, shell, surface, and structural proteins (e.g., VP1, VP2). A VLP may also resemble the structure of a bacteriophage, being non-replicative and noninfectious, and lacking at least the gene or genes coding for the replication machinery of the bacteriophage, and also lacking the gene or genes encoding the protein or proteins responsible for viral attachment to or entry into the host.

### Vectors

The vectors useful for the present disclosure are suitable for replication and, optionally, integration in eukaryotic cells. Typical vectors contain transcription and translation terminators, initiation sequences, and promoters useful for regulation of the expression of the desired nucleic acid sequence.

The vectors of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

In some embodiments, an isolated nucleic acid of interest can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

In some embodiments, the vector may be provided to a cell in the form of a viral vector. Viral vector technology is well known in the art. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells (e.g., BHK, VERO, HT1080, 293, RD, COS-7, or CHO cells). For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

In some embodiments, the viral vector can be any vector including, but not limited to, adeno-associated virus (“AAV”), recombinant adeno-associated virus (“rAAV”), parvovirus, dependoviruses, adenovirus, lentivirus, SV40 virus or combinations thereof. In some embodiments, the viral vector is linear.

In some embodiments, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In some embodiments, the viral vector is an AAV vector or any variant and serotype thereof, for example AAV1, AAV2, AAV3, AAV4, AAV5, AAV6, AAV7, AAV8, and or AAV9. In some embodiments, the AAV is AAV2, AAV5, AAV8, and/or AAV9. The viral vector can comprise a reporter such as a fluorescent or chemiluminescent reporter, for example, green fluorescent protein (GFP) and luciferase, respectively.

AAV vectors provide powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Gene therapy compositions can include viral vectors, including adenovirus associated vectors (AAV), and variants and serotypes thereof, for example AAV1, AAV2, AAV3, AAV4, AAV5, AAV6, AAV7, AAV8, and or AAV9. Expression of a particular gene contained within an AAV vector can be specifically targeted to one or more types of cells by choosing the appropriate combination of AAV serotype, promoter, and delivery methods.

In one embodiment, the gene of interest is contained within an AAV vector. More than 30 naturally occurring serotypes of AAV are available. Many natural variants in the AAV capsid exist, allowing identification and use of an AAV with properties specifically suited for skeletal muscle. AAV viruses may be engineered using conventional molecular biology techniques, making it possible to optimize these particles for cell specific delivery of myotubularin nucleic acid sequences, for minimizing immunogenicity, for tuning stability and particle lifetime, for efficient degradation, for accurate delivery to the nucleus, etc.

In some embodiments, the transposase includes without limitation, Sleeping Beauty (e.g., Sleeping Beauty 10, 11, 100X), Piggyback, Piggy bat, Mu (e.g., MuA), TcBuster, Mos1, Tol1, Tol2, Frog Prince, spinON, HimarI, Passport, Minos, hAT, Hsmar1, Harbinger, Harbinger3-DR, AciDs, PIF, Tn3, Tn5, Tn10, Tn552, Tn903, Ty1, Tc1, IS5, IS911, Mariner and derivatives and analogues thereof. Also included are combinations of transposases. As used herein, transposons are those that are readily recognized by and available to those skilled in the art. In some embodiments, the transposon corresponds to its respective transposase, for example a Sleeping Beauty transposon or a PiggyBac transposon. In some embodiments, the construct and viral vector comprise a promotor.

Some embodiments provided herein relate to single component constructs and viral vectors for delivery of nucleic acids into a target cell genome comprising:
a) a first nucleic acid encoding a transposon comprising a gene of interest; and b) a second nucleic acid encoding a transposase that is functional in target cells and not functional in producer cells.

In some embodiments, methods of virus production are described wherein a nonmammalian (e.g., invertebrate) production host (e.g., producer cells) is utilized to manufacture the viral particles. The construct of interest described herein can be delivered to production cells by standard methods of transduction known to those skilled in the art using viruses compatible with the production cells, e.g., baculoviruses. The difference between spliceosomes and splice site recognition between the species, here target cells (i.e., mammalian cells) and producer cells (i.e., non-mammalian cells (e.g., insect cells such as Trichoplusia ni (Tn5) or Sf9)) is utilized herein to allow for the production of the vector without premature transposase activity in the production cells. Premature transposase activity in the production cells leads to destruction of the vector genome before it can be packaged into a viral particle.

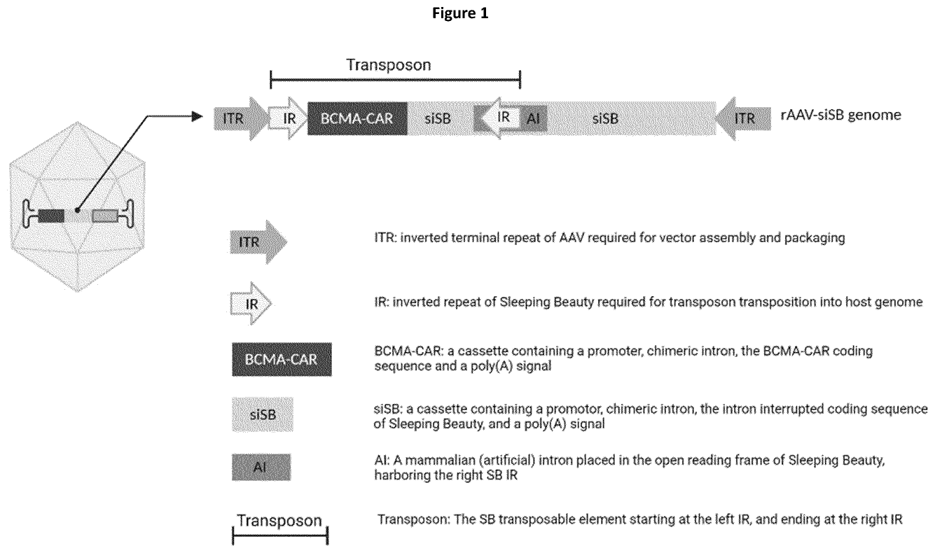

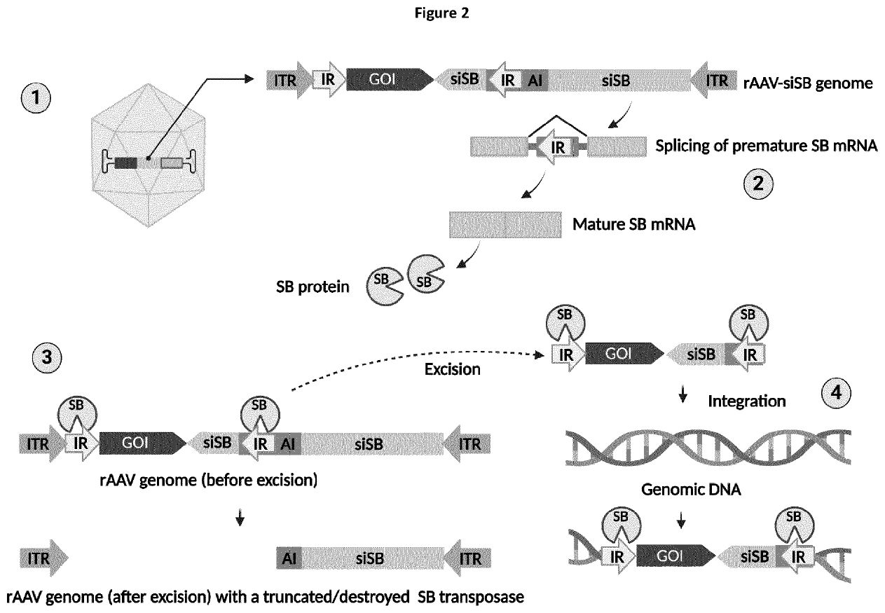

In some embodiments, both ends of the transposon are flanked by inverted-repeat transposable elements recognized specifically by the transposase. In some embodiments, inverted terminal repeats flank a gene of interest, part of a transposase on a lagging strand and part of the intron. All four pieces (the gene of interest, the inverted terminal repeat on either end of the gene of interest, part of the transposase on a lagging strand and part of the intron) make up a transposon region (for clarity see FIG. 1). In some embodiments, the transposon/transposase system of the construct and viral vector is flanked by transposon inverted repeats (IR's) together with a transposase, with the entire cassette being flanked by AAV inverted terminal repeats (ITRs) (FIG. 1). In some embodiments, the viral vector is utilized to deliver a gene of interest to a target cell, wherein the viral vector comprises: a) a first nucleic acid which encodes a transposon comprising a gene of interest and b) a second nucleic acid which encodes a transposase that is functional in target cells and not functional in producer cells. This viral vector, due to its design, is capable of integrating into the genome of the subjected mammalian cell, herein called “target cells” and expressing its intended cargo (e.g., gene of interest), while at the same time partially or completely inactivating or destroying the transposase open reading frame, thereby partially or completely inhibiting the generation of transposase protein (FIG. 2). This inactivation or destruction of transposase is a designed-in safety feature of the vector, as it provides self-limitation of the transposase upon transposition.

In some embodiments, the cargo is one or more therapeutic agents, including therapeutic agents described herein. In some embodiments, exemplary polypeptides, nucleic acids, or other therapeutic agents include those useful in the treatment of rare sarcoglycanopathies and dystrophinopathies like Duchenne muscular dystrophy, limb girdle muscle disease, and spinal muscular atrophy, as well as other muscle tissue related diseases. Exemplary muscle tissue related diseases include but are not limited to Acid Maltase Deficiency (AMD), Amyotrophic Lateral Sclerosis (ALS), Andersen-Tawil Syndrome, Becker Muscular Dystrophy (BMD), Becker Myotonia Congenita, Bethlem Myopathy, Bulbospinal Muscular Atrophy (Spinal-Bulbar Muscular Atrophy), Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Muscular Dystrophy (CMD), Congenital Myasthenic Syndromes (CMS), Congenital Myotonic Dystrophy, Cori Disease (Debrancher Enzyme Deficiency), Debrancher Enzyme Deficiency, Dejerine-Sottas Disease (DSD), Dermatomyositis (DM), Distal Muscular Dystrophy (DD), Duchenne Muscular Dystrophy (DMD), Dystrophia Myotonica (Myotonic Muscular Dystrophy), Emery-Dreifuss Muscular Dystrophy (EDMD), Endocrine Myopathies, Eulenberg Disease (Paramyotonia Congenita), Facioscapulohumeral Muscular Dystrophy (FSH or FSHD), Finnish (Tibial) Distal Myopathy, Forbes Disease (Debrancher Enzyme Deficiency), Friedreich's Ataxia (FA), Fukuyama Congenital Muscular Dystrophy, Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Kennedy Disease (Spinal-Bulbar Muscular Atrophy), Kugelberg-Welander Disease (Spinal Muscular Atrophy), Lactate Dehydrogenase Deficiency, Lambert-Eaton Myasthenic Syndrome (LEMS), Limb-Girdle Muscular Dystrophy (LGMD), Lou Gehrig's Disease (Amyotrophic Lateral Sclerosis), McArdle Disease (Phosphorylase Deficiency), Merosin-Deficient Congenital Muscular Dystrophy, Metabolic Diseases of Muscle, Mitochondrial Myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphoglycerate Kinase Deficiency, Phosphoglycerate Mutase Deficiency, Phosphorylase Deficiency, Phosphorylase Deficiency, Polymyositis (PM), Pompe Disease (Acid Maltase Deficiency), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia COngenita), Ullrich Congenital Muscular Dystrophy, Walker-Warburg Syndrome (Congenital Muscular Dystrophy), Welander Distal Myopathy, Werdnig-Hoffmann Disease (Spinal Muscular Atrophy), and ZASP-Related Myopathy.

In some embodiments, exemplary polypeptides include neuroprotective polypeptides and anti-angiogenic polypeptides. Suitable polypeptides include, but are not limited to, glial derived neurotrophic factor (GDNF), fibroblast growth factor 2 (FGF-2), nurturin, ciliary neurotrophic factor (CNTF), nerve growth factor (NGF; e.g., nerve growth factor-.beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Flt-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic products encoded by the heterologous nucleic acid sequence include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful heterologous nucleic acid sequence products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful heterologous nucleic acid sequence products include any one of the receptors for the hormones, growth factors, cytokines, lymphokines, regulatory proteins and immune system proteins. Useful heterologous nucleic acid sequences also include receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4 C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful heterologous nucleic acid sequence products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione betasynthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence. Still other useful gene products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding β-glucuronidase (GUSB)).

In some embodiments, useful heterologous nucleic acid sequence products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). The Factor VIII gene codes for 2351 amino acids and the protein has six domains, designated from the amino to the terminal carboxy terminus as A1-A2-B-A3-C1-C2 (Wood et al., (1984) Nature, 312:330; Vehar et al., (1984) Nature 312:337; and Toole et al., (1984) Nature, 342:337). Human Factor VIII is processed within the cell to yield a heterodimer primarily comprising a heavy chain containing the A1, A2 and B domains and a light chain containing the A3, C1 and C2 domains. Both the single chain polypeptide and the heterodimer circulate in the plasma as inactive precursors, until activated by thrombin cleavage between the A2 and B domains, releasing the B domain and results in a heavy chain consisting of the A1 and A2 domains. The B domain is deleted in the activated procoagulant form of the protein. Additionally, in the native protein, two polypeptide chains (“a” and “b”), flanking the B domain, are bound to a divalent calcium cation.

In some embodiments, useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, used to reduce overexpression of a target.

In some embodiments, the present invention provides methods for treatment of a stem cell disorder, for example a disorder in either bone marrow stem cells or adult tissue stem cells (i.e., somatic stem cells). In some embodiments, adult stem cells can include organoid stem cells (i.e., stem cells derived from any organ or organ system of interest within the body). Organs of the body include for example but are not limited to skin, hair, nails, sense receptors, sweat gland, oil glands, bones, muscles, brain, spinal cord, nerve, pituitary gland, pineal gland, hypothalamus, thyroid gland, parathyroid, thymus, adrenals, pancreas (islet tissue), heart, blood vessels, lymph nodes, lymph vessels, thymus, spleen, tonsils, nose, pharynx, larynx, trachea, bronchi, lungs, mouth, pharynx, esophagus, stomach, small intestine, large intestine, rectum, anal canal, teeth, salivary glands, tongue, liver, gallbladder, pancreas, appendix, kidneys, ureters, urinary bladder, urethra, testes, ductus (vas) deferens, urethra, prostate, penis, scrotum, ovaries, uterus, uterine (fallopian) tubes, vagina, vulva, and mammary glands (breasts). Organ systems of the body include but are not limited to the integumentary system, skeletal system, muscular system, nervous system, endocrine system, cardiovascular system, lymphatic system, respiratory system, digestive system, urinary system, and reproductive system. In some embodiments, the disorder for treatment is a disorder in any one or more organoid stem cells (i.e., stem cells derived from any organ or organ system of interest within the body).

The delivery particles described herein may be used and further comprise a number of different cargo molecules for delivery. Representative cargo molecules may include, but are not limited to, nucleic acids, polynucleotides, proteins, polypeptides, polynucleotide/polypeptide complexes, small molecules, sugars, or a combination thereof. Cargoes that can be delivered in accordance with the systems and methods described herein include, but are not necessarily limited to, biologically active agents, including, but not limited to, therapeutic agents, imaging agents, and monitoring agents. A cargo may be an exogenous material or an endogenous material.

Biologically active agents include any molecule that induces an effect in a cell. Biologically active agents may be a protein, a nucleic acid, a small molecule, a carbohydrate, and a lipid. When the cargo is or comprises a nucleic acid, the nucleic acid may be a separate entity from the DNA-based carrier. In these embodiments, the DNA-based carrier is not itself the cargo. In other embodiments, the DNA-based carrier may itself comprise a nucleic acid cargo. Therapeutic agents include chemotherapeutic agents, anti-oncogenic agents, antiangiogenic agents, tumor suppressor agents, anti-microbial agents, enzyme replacement agents, gene expression modulating agents and expression constructs comprising a nucleic acid encoding a therapeutic protein or nucleic acid. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

In some embodiments, the cargo comprises one or more nucleic acid molecules selected from DNA, mRNA, tRNA, rRNA, siRNA, microRNA, regulating RNA, and non-coding and coding RNA.

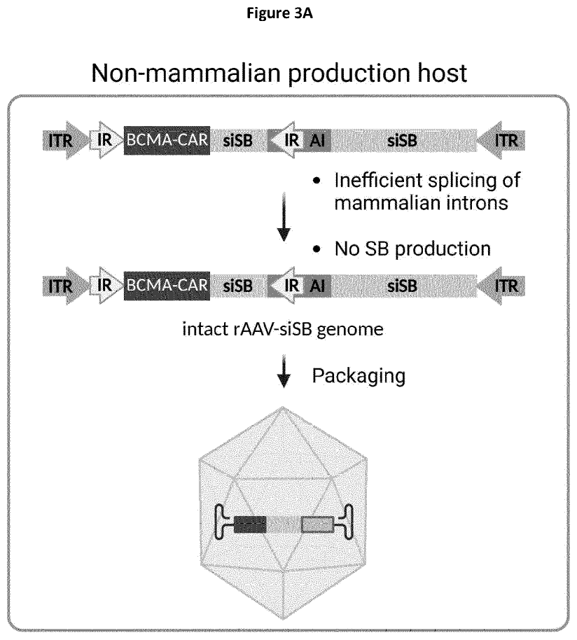

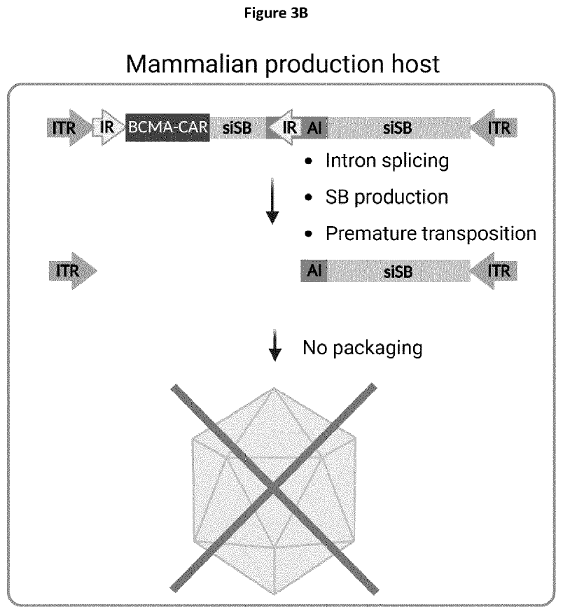

In order to produce the disclosed viral vector, however, a production system is needed where the self-inactivating feature of the viral vector is silent in the producer cell. That is, the transposase is not functional in the producer cells. Otherwise, the transposase would self-destruct during the production process in the producer cell. In one embodiment, the present disclosure describes the use of a production system for the viral vector, e.g., for AAV and other viruses, for transposon/transposase systems in combination with introns that are differentially spliced (i.e., spliced and functional in mammalian target cells and not spliced and functional in non-mammalian producer cells). The viral vector comprises a transposase that is functional in target cells but not functional in producer cells, where introns are unspliced. Lack of splicing in producer cells will not allow for transposase expression in producer cells, and therefore no expression of the transposase will occur, which results in appropriate generation of the viral vector (FIGS. 3A-3B).

In some embodiments, The transposase can be a Tn transposase (e.g. Tn3, Tn5, Tn7, Tn10, Tn552, Tn903), a transposase fused with protein A, protein G (e.g., pAG-Tn5) a MuA transposase, a Vibhar transposase (e.g. from Vibrio harveyi), Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Tel, THE-1, Tn/O, TnA, Tn3, Tn5, Tn7, Tn10, Tn552, Tn903, Tol1, Tol2, TnlO, Tyl, any prokaryotic transposase, or any transposase related to and/or derived from those listed above. In some embodiments, the tagmentation comprises a fusion of protein A, protein G, and Tn5 transposase (pAG-Tn5).

In some embodiments, the constructs and viral vectors comprise a first nucleic acid encoding a transposon comprising a gene of interest (GOI), wherein the transposon also comprises, but not limited to, a promotor, a drug selection gene or other components. In some embodiments the GOI is flanked by inverted terminal repeats.

With reference to the Examples and Figures, in some embodiments, the constructs and viral vectors comprise or consist essentially of a) a first cassette comprising or consisting essentially of, e.g., in sequential order, an inverted repeat of a transposon, a CMV enhancer+promoter, an intron (e.g., a chimeric intron), a gene of interest coding sequence and a poly(A) signal (e.g., a bGH poly(A) signal; and b) a second cassette, e.g., downstream of the first cassette, on the lagging strand and in reverse orientation, comprising or consisting essentially of, in sequential order, a promoter (e.g., a SV40 promoter), a first intron (e.g., a SV40 intron), a first part of a first amino acid coding sequence of a transposase (preferentially the coding sequence of the first 92 amino acids (from the N-terminus) of SB100X), a first part of a second intron (e.g., a mammalian intron, a human intron, TP53 or GH), a transposase specific inverted repeat (e.g., the right specific inverted repeat, in reverse orientation in respect of the orientation of the cassette, the right Sleeping Beauty specific inverted repeat in the reverse orientation), a second part of the second intron (e.g., a human intron, TP53 or GH), a second part of the first amino acid coding sequence of the transposase (e.g., the remaining amino acid coding sequence of the first amino acid coding sequence of the transposase, e.g., the coding sequence of 249 amino acids of SB100X), and a poly(A) signal (e.g., a SV40 poly(A) signal).

In some embodiments the first and second cassettes are flanked by inverted terminal repeats of a virus (e.g., AAV).

In some embodiments, the coding sequence of the transposase (e.g., SB100X) is interrupted by an intron (e.g., a mammalian intron, or a human intron and in which the right transposase (e.g., SB100X) inverted repeat is placed). In some embodiments, the mammalian intron allows for expression of the transposase in target cells (e.g., human cells), but does not allow for expression in producer cells (e.g., insect cells).

In some embodiments, the mammalian intron is recognized by a mammalian spliceosome, but not recognized by a non-mammalian spliceosome.

In some embodiments, the mammalian intron is removed during RNA processing in target cells, thereby restoring the transposase (e.g., SB100X) coding sequence/reading frame and translation of the transposase (e.g., SB100X transposase) in target cells. Preferably, the mammalian intron is not removed during RNA processing in producer cells, thereby interrupting the coding sequence of the transposase by the mammalian intron in the producer cells (e.g., insect cells), and prematurely ending translation of the transposase in the producer cells. In some embodiments, the mammalian intron comprises one or more stop codons (e.g., the stop codons are positioned early on in the intron sequence). In some embodiments, the transposase (e.g., SB100X) is not expressed in producer cells (e.g., insect cells). In some embodiments, the transposition of the transposon does not occur in producer cells and the viral genome is left intact and packaged into viral particles. In some embodiments, the transposase gene (e.g., SB100X gene) comprises a mammalian intron which allows the right transposase (e.g., SB) inverted repeat to be introduced inside the transposase gene (e.g., SB100X gene) without blocking expression of the transposase (e.g., SB100X) and this intron is spliced out during RNA processing in target cells. In some embodiments, the right transposase (e.g., SB) inverted repeat is positioned in the coding sequence of the transposase (e.g., SB100X), thereby ensuring its own inactivation upon initial episomal transposase (e.g., SB100X) expression and subsequent transposition of the transposon. In some embodiments, the promotor (e.g., SV40) and/or intron (e.g., SV40) and/or the first transposase codons (e.g., first 92 SB100X codons) do not integrate into the producer cell genome. In some embodiments, the transposase (e.g., SB100X) open reading frame is abrogated.

Introduction or delivery of constructs into producer cells (e.g., insect cells) can be by any means known by those skilled in the art, including without limitation, baculovirus transduction, electroporation, non-viral delivery. In some embodiments, target cells and/or producer cells are transfected/nucleofected by lipofectamine/epifectamine transfection or nucleofection.

In some embodiments, the self-integrating and self-inactivating transposase (e.g., siSB) integrates into the target cell genome. In other embodiments, the construct and viral vector comprise a mammalian intron (e.g., TP53 intron). In some embodiments, the mammalian intron is not recognized by producer cells (e.g., insect cells). In yet other embodiments, the viral vector is produced in producer cells (e.g., insect cells) without expression of the transposase protein (e.g., SB100X).

In some embodiments described herein, the constructs and viral vector comprise a first nucleic acid encoding a transposon comprising a gene of interest (GOI), wherein the GOI includes, but is not limited to, CAR CD19, BCMA, CD20, HER-2, CEA, CD7, CD22, CD33, CD44v6, CD123, CD135, CD38, CD138, CD269, CD319, ROR1, ROR2, GD2, EGFR and its variants, EpCAM, GPRC5D, PSMA, WT1, PSCA, ERBB2, CD133, L1 CAM, extracellular domain of MUC16 (MUC-CD), mesothelin, CEA, CD24, carboxy-anhydrase-IX (CAIX), Slamf7, FLT3, Siglec-6, aₙb₃ integrin, FAP, CD5, NY-ESO-1, MAGEA3 and MAGEA4.

In some embodiments, the constructs and viral vectors comprise a first nucleic acid encoding a transposon comprising a gene of interest (GOI), wherein the GOI includes, but is not limited to, Factor 9 (F9), Factor 8 (F8), Alanine:Glyoxylate-aminotransferase (AGXT), Alpha-L-iduronidase (IDUA), Ornithine Transcarbamylase (OTC), Lysosomal acid lipase (LIPA), argininosuccinate synthase 1 (ASS1), argininosuccinate lyase (ASL), galactosidase alpha (GLA), cathepsin A (CTSA), arylsulfatase B (ARSB), glucosylceramidase beta 1 (GBA), iduronate 2-sulfatase (IDS), cystathionine beta-synthase (CBS), acyl-CoA synthetase family member 3 (ACSF3), methylmalonyl-CoA mutase (MMUT), propionyl-CoA carboxylase subunit alpha (PCCA), propionyl-CoA carboxylase subunit beta (PCCB), glucose-6-phosphatase catalytic subunit 1 (G6PC), branched chain keto acid dehydrogenase E1 subunit alpha (BCKDHA), phenylalanine hydroxylase (PAH), UDP glucuronosyltransferase family 1 member A1(UGT1A1), N-sulfoglucosamine sulfohydrolase (SGSH), galactosamine (N-acetyl)-6-sulfatase (GALNS), prosaposin (PSAP), hexosaminidase subunit alpha (HEXA), mucolipin TRP cation channel 1 (MCOLN1), sphingomyelin phosphodiesterase 1 (SMPD1), galactosylceramidase (GALC), galactosidase beta 1 (GLB1), 1,4-alpha-glucan branching enzyme 1 (GBE1), N-acetyl-alphaglucosaminidase (NAGLU), hydroxyacyl-CoA dehydrogenase trifunctional multienzyme complex subunit alpha (HADHA), ATP binding cassette subfamily D member 1 (ABCD1), interleukin 12 receptor subunit beta 1 (IL12RB1), acyl-CoA dehydrogenase very long chain (ACADVL), heparan-alpha-glucosaminide N-acetyltransferase (HGSNAT), hexosaminidase subunit beta (HEXB), serpin family A member 1 (SERPINA1), survival motor neuron 1 (SMN1), cystinosin, lysosomal cystine transporter (CTNS), wherein such first nucleic acid is flanked by transposon inverted repeats and wherein the vector also comprises a second nucleic acid encoding a non-functional transposase that is activated in target cells but not activated in producer cells, wherein the transposase includes, but is not limited to, Sleeping Beauty 10, 11, 100X, PiggyBac, Piggy bat, Mu, TcBuster, Mos1, Tol1, Tol2, Frog Prince, spinON, HimarI, Passport, Minos, hAT, Hsmar1, Harbinger, Harbinger3-DR, AciDs, PIF. In some embodiments the constructs and viral vectors are flanked by inverted terminal repeats of the virus used for the delivery into mammalian cells.

In some embodiments the constructs and viral vectors comprise a promoter region where promoter includes, but is not limited to, EF1a, CMV, SV 40, where the sequence comprises a nucleic acid sequence comprising a homology of at least 85%, 86%, 87%, 88%, 89%, 90%, 91%, 92%, 93%, 94%, 95%, 96%, 97%, 98%, 99% or 100% to SEQ ID NO: 8, SEQ ID NO: 10.

In some embodiments the constructs and viral vectors comprise a polyadenylation signal where polyA includes without limitation a synthetic polA, bgH, SV40 polyA, where the sequence comprises a nucleic acid sequence comprising a homology of at least 85%, 86%, 87%, 88%, 89%, 90%, 91%, 92%, 93%, 94%, 95%, 96%, 97%, 98%, 99% or 100% to SEQ ID NO: 14. In some embodiments, the polyA signal comprises combinations with other elements of the constructs and viral vectors.

In some embodiments, the constructs and viral vectors further comprise a sequence encoding a suicide gene. In some embodiments, the suicide gene is thymidine kinase, oxidoreductase, cytosine deaminase, thymidine kinase thymidylate kinase (Tdk::Tmk), or deoxycytidine kinase.

In some embodiments, the constructs and viral vectors comprise a drug selection gene. In some embodiments, the drug selection gene encodes dihydrofolate reductase (DHFR), DHFR double mutant (DHFRdm), hugromycin-B phosphotransferase (hph), aminoglycoside phosphotransferase, beta-lacatamase, chloramphenicol acetyl-transferase (CAT), adenosine deaminase (ADA), thymidine kinase (TK), lacz (encoding beta-galactosidase), bleomycin resistance, metallothionein, or xanthine guanine phosphoribosyltransferase (XGPRT). In some embodiments, the agent for selection is methotrexate.

In some embodiments the constructs and viral vectors comprise a transposase sequence, wherein the transposase is Sleeping Beauty 100× comprising an intron, wherein the sequence comprises a nucleic acid sequence comprising a homology of at least 85%, 86%, 87%, 88%, 89%, 90%, 91%, 92%, 93%, 94%, 95%, 96%, 97%, 98%, 99% or 100% to SEQ ID NO: 7.

In some embodiments the constructs and viral vectors comprise an intron where intron is selected from TP53 intron 1, TP53 intron 2, Chimeric intron, β-globin/IgG chimeric intron, EF1a intron, CMV intron or SV40. In some embodiments the intron comprises a sequence homology of at least 85%, 86%, 87%, 88%, 89%, 90%, 91%, 92%, 93%, 94%, 95%, 96%, 97%, 98%, 99% or 100% to SEQ ID NOs: 5, 6, or 13. In some embodiments the intron is positioned within the open reading frame of transposase as shown in SEQ ID NO: 7. In some embodiments the intron is selected to be functional in target cells but not in producer cells used for virus production. In some embodiments, the intron comprises combinations with other elements of the constructs or viral vectors. In some embodiments, the intron is positioned within any position of the transposase.

In some embodiments, the target cells include, but are not limited to, human cells, primary cells, such as a T-cell, a human T-cell, or a stem cell, preferably a human stem cell.

In some embodiments the viral vector is generated (e.g., produced) in producer cells. In some embodiments the producer cells include, but are not limited to, insect cells, SF9, SF+, yeasts cells Saccharomyces, Pichia pastori, procarotic cells, E. coli lactococus lactis.

In certain embodiments the constructs and viral vectors comprise a chimeric antigen receptor (CAR). In some embodiments the viral vector expresses a chimeric antigen receptor. CAR refers to a chimeric synthetic receptor engineered to be expressed on the target cells and to bind to a specific antigen. CARs are known to those skilled in the art and include, but are not limited to, CAR CD19, BCMA, CD20, HER-2, CEA, CD7, CD22, CD33, CD44v6, CD123, CD135, CD38, CD138, CD269, CD319, ROR1, ROR2, GD2, EGFR and its variants, EpCAM, GPRC5D, PSMA, WT1, PSCA, ERBB2, CD133, L1 CAM, extracellular domain of MUC16 (MUC-CD), mesothelin, CEA, CD24, carboxy-anhydrase-IX (CAIX), Slamf7, FLT3, Siglec-6, aₙb₃ integrin, FAP, CD5, NY-ESO-1, MAGEA3, MAGEA4.

In certain embodiments, the constructs and viral vectors comprise a gene of interest that encodes a therapeutic protein. In some embodiments the viral vector expresses a therapeutic protein in-vitro or in vivo. Therapeutic proteins are known to those skilled in the art and include, but are not limited to, Factor 9 (F9), Factor 8 (F8), Alanine:Glyoxylate-aminotransferase (AGXT), Alpha-L-iduronidase (IDUA), Ornithine Transcarbamylase (OTC), Lysosomal acid lipase (LIPA), argininosuccinate synthase 1 (ASS1), argininosuccinate lyase (ASL), galactosidase alpha (GLA), cathepsin A (CTSA), arylsulfatase B (ARSB), glucosylceramidase beta 1 (GBA), iduronate 2-sulfatase (IDS), cystathionine beta-synthase (CBS), acyl-CoA synthetase family member 3 (ACSF3), methylmalonyl-CoA mutase (MMUT), propionyl-CoA carboxylase subunit alpha (PCCA), propionyl-CoA carboxylase subunit beta (PCCB), glucose-6-phosphatase catalytic subunit 1 (G6PC), branched chain keto acid dehydrogenase E1 subunit alpha (BCKDHA), phenylalanine hydroxylase (PAH), UDP glucuronosyltransferase family 1 member A1(UGT1A1), N-sulfoglucosamine sulfohydrolase (SGSH), galactosamine (N-acetyl)-6-sulfatase (GALNS), prosaposin (PSAP), hexosaminidase subunit alpha (HEXA), mucolipin TRP cation channel 1 (MCOLN1), sphingomyelin phosphodiesterase 1 (SMPD1), galactosylceramidase (GALC), galactosidase beta 1 (GLB1), 1,4-alpha-glucan branching enzyme 1 (GBE1), N-acetyl-alpha-glucosaminidase (NAGLU), hydroxyacyl-CoA dehydrogenase trifunctional multienzyme complex subunit alpha (HADHA), ATP binding cassette subfamily D member 1 (ABCD1), interleukin 12 receptor subunit beta 1 (IL12RB1), acyl-CoA dehydrogenase very long chain (ACADVL), heparan-alpha-glucosaminide N-acetyltransferase (HGSNAT), hexosaminidase subunit beta (HEXB), serpin family A member 1 (SERPINA1), survival motor neuron 1 (SMN1), cystinosin, lysosomal cystine transporter (CTNS).

In some embodiments, the disclosed delivery vehicle are useful in conjunction with the CRISPR/Cas system for gene editing. In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term Is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In some embodiments, ARCUS nuclease is considered for the disclosed delivery vehicle for gene editing. ARCUS nucleases (Precision BioSciences) are based on a naturally occurring genome editing enzyme, I-CreI, a homing endonuclease that evolved in the algae Chlamydomonas reinhardtii to make highly specific cuts and DNA insertions in cellular DNA. The nuclease is able to deactivate itself once gene edits are made, which minimizes potential off-targeting.

In certain embodiments, the viral vector can be used to engineer target cells to stably express a GOI.

In some embodiments the viral vector is used to express a GOI in animals or humans for therapeutic purposes.

In some embodiments, the disclosed composition (e.g., viral vectors) and methods and are useful as a therapeutic intervention that ameliorates a sign or symptom of a disease or pathological condition, such a sign or symptom of cancer. Treatment can also induce remission or cure of a condition, such as cancer and in particular a central nervous system (CNS) cancer or tumor. In particular examples, treatment includes preventing a disease, for example by inhibiting the full development of a disease, such as preventing development of tumor metastasis. Prevention of a disease does not require a total absence of a dysplasia or cancer. For example, a decrease of at least about 50% can be sufficient.

The various alternatives described herein are not meant to be limiting. Other alternatives may be utilized without departing from the scope of the present disclosure described herein. As such, one skilled in the art would readily understand that the aspects of the present disclosure generally described herein can be arranged, substituted, combined and designed in a wide variety of different configurations, all of which are explicitly contemplated herein. Non-limiting examples include various arrangements and combinations of transposons, transposases, introns, GOIs, polyAs, promoters, and other genetic elements are contemplated herein.

### EXAMPLES

### 1. Example 1: Integration and Expression of the Vector

In order to demonstrate the correct integration and expression of the vector, two constructs were generated; one (i) with a BCMA-CAR cassette and an additional cassette which encodes a functional Sleeping Beauty transposase (this construct is also known as BCMA-siSB), and (ii) a control plasmid that is identical to plasmid (i) except for two nucleotides in the SB coding sequence that negates SB expression (BCMA-siSB-scrambled). Both constructs contained two separate cassettes. The first cassette (identical in both constructs, i and ii) contains the following elements in a sequential order:
a. The left SB specific inverted repeat
b. The CMV enhancer+promotor
c. A chimeric intron
d. The BCMA coding sequence
e. The bGH poly(A) signal

The second cassette of construct (i) is downstream of the first cassette, but on the lagging strand, and therefore in the reverse orientation, which contains the following elements in sequential order (5′ to 3′);
f The SV40 promoter
g. The SV40 intron
h. The coding sequence of the first 92 amino acids (from N-terminus) of SB100X
i. The first part of a human intron (TP53, or GH)
j. The right SB specific inverted repeat (in the reverse orientation in respect to the orientation of the cassette)
k. The second part of the human intron (TP53, or GH)
l. The remaining coding sequence of 249 amino acids of SB100X
m. The SV40 poly(A) signal

The sequence of second cassette in plasmid (ii) is identical to the second cassette on plasmid (i), but for 2 nucleic acid bases. These two bases changes triggered the Tyrosine codon on the 56^{th} position, and the Serine codon on 58^{th} position of the amino acids sequence to change into stop codons. These premature stop codons can halt the translation of SB100X, not producing any (functional) transposase, and hence be referred to as the scrambled controls.

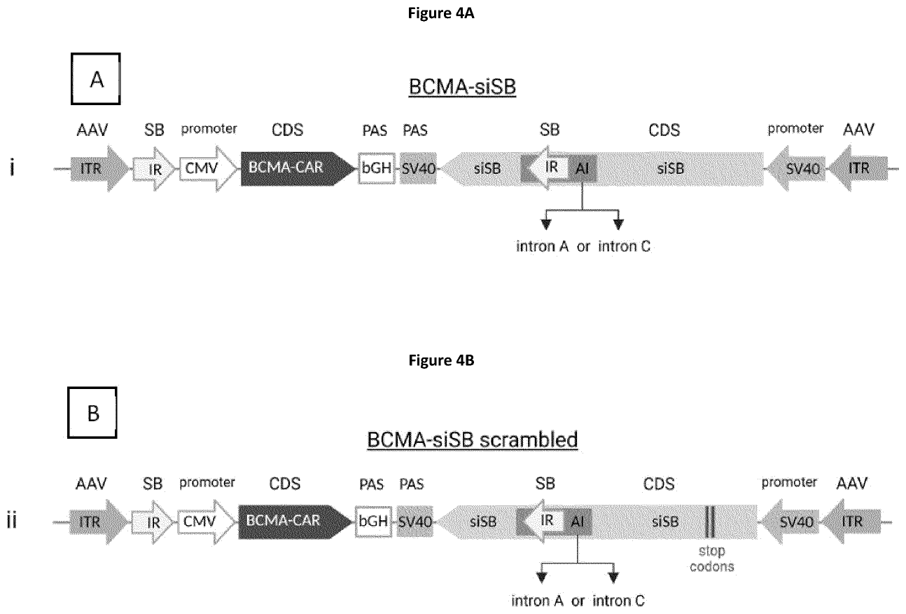

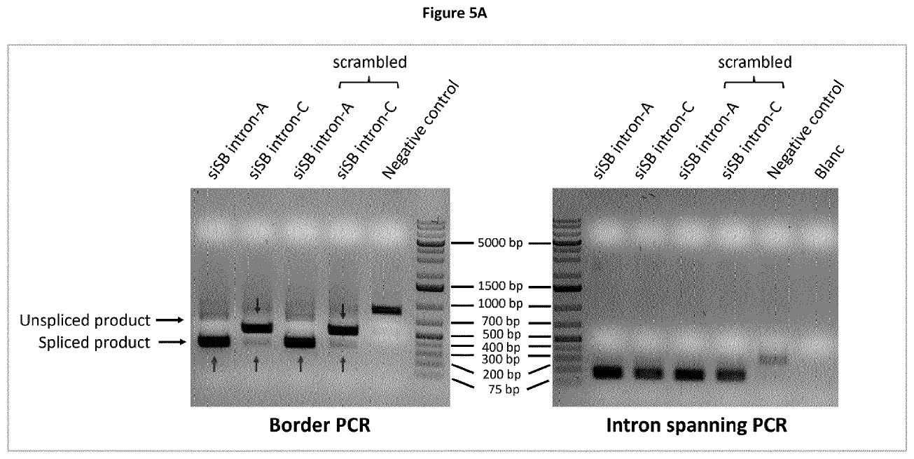

These two cassettes are, furthermore, flanked by inverted terminal repeats of the virus, here AAV, which thus encompasses the full two cassettes in a AAV genome. FIG. 1 and FIGS. 4A-4B provide schematic overviews of the custom AAV genome and all the previously summarized elements in order. As can be deduced from the summary of elements, and seen in FIG. 1, the SB100X gene has its coding sequence interrupted by a mammalian (e.g., human) intron, in which the right SB inverted repeat is placed. The sequence between the two SB inverted repeats (the left at the beginning of the first cassette, and the right in an intron, within the SB100X gene), constitutes the transposable element (FIG. 1). The mammalian intron allows for SB100X expression in mammalian cells, in contrast to insect cells, as this intron is recognized by the mammalian spliceosome, but not by the insect spliceosome. Removal of this intron during RNA processing restores the SB100X coding sequence/reading frame in mammalian cells and can therefore be translated into SB100X transposase (FIG. 2). Without the removal of the intron, the coding sequence is interrupted by the intron, and translation will prematurely end due to the presence of stop codons early on in the intron sequence. SB100X is therefore not expressed in insect cells, and transposition of the transposon is thus not occurring, which leaves the AAV genome intact to be packaged into a viral particle. Because mammalian cells do allow for the expression of SB100X transposase, these cells cannot be used as a production host, as the AAV genome will be destroyed by the transposition events upon SB100X expression (FIGS. 3A-3B). The presence of the mammalian intron in the SB100Xx gene thus allowed introducing the right SB inverted repeat inside the SB100X gene, without blocking SB100X expression, as this intron is spliced out during RNA processing. The positioning of the right SB inverted repeat, in the coding sequence of SB100X, ensures its own inactivation upon initial episomal SB100X expression and the subsequent transposition of the transposon, as the SV40 promotor (and SV40 intron) and first 92 SB100X codons will not integrate into the host genome, thus abrogating the SB100X open reading frame (FIG. 2).

The self-inactivating SB technology was constructed and tested with two different human introns, A (a TP53 intron) and intron C (a hGH intron) (FIGS. 4A-4B).

### 2. Example 2: Validation of Construct Validity

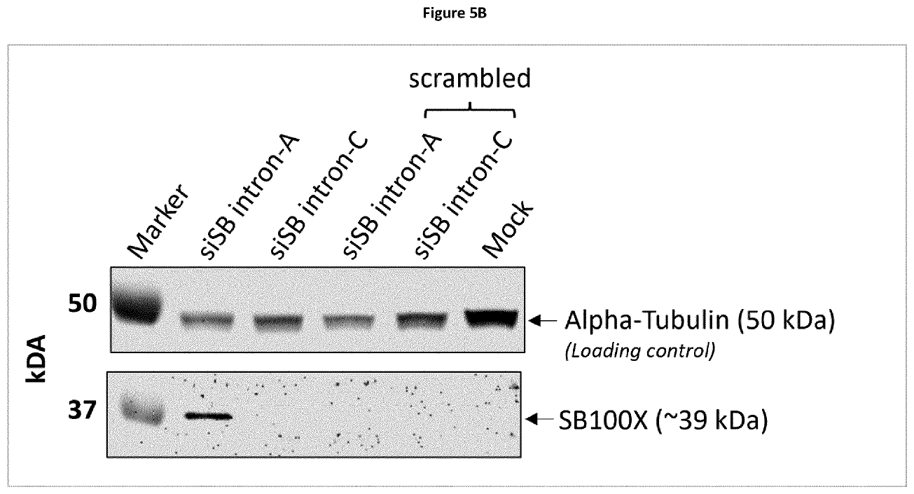

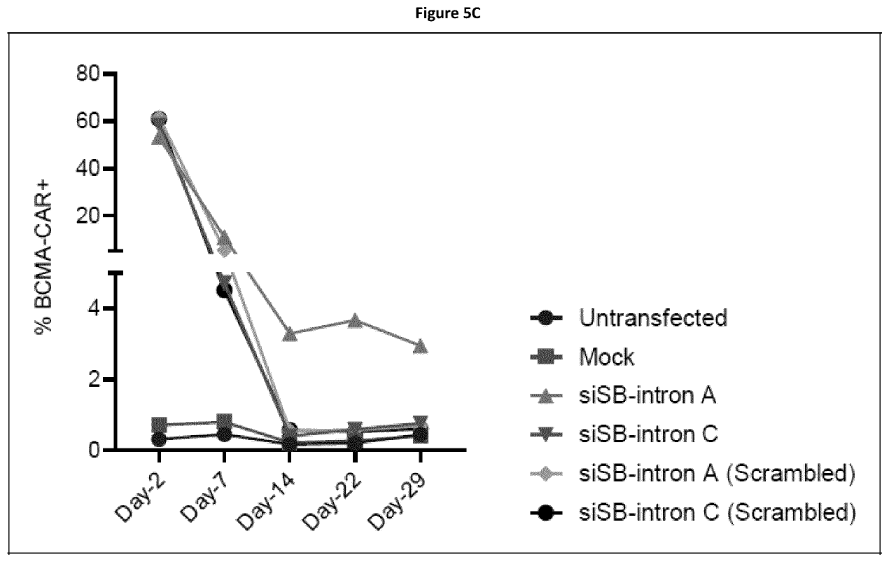

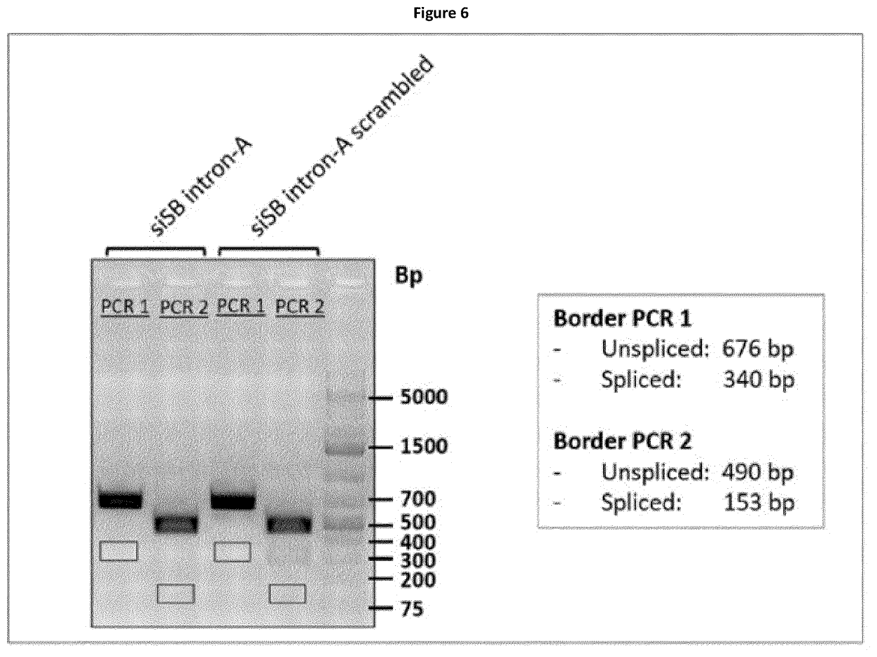

To validate correct functionality of the constructs in mammalian cells (i.e., target cells), and in insect cells (i.e. AAV producing cells), the disclosed constructs were tested in the HEK293t cell line (mammalian cells) and SF9 cells (insect cells). As mentioned earlier, the artificial introns that were placed in the SB100X coding sequence are of mammalian origin. It was hypothesized that correct splicing of the SB100X transcript and subsequent transposase production in the mammalian cells would occur, that no splicing event would occur in the insect cells of the SB100X transcript, and that therefore no SB100X protein will be found in the SF9 insect cells. The siSB constructs were introduced into the HEK293t and SF9 cells by either lipofectamine/epifectamine transfection, or nucleofection. Cells were allowed to recover and grow for 48-72 hours after transfection/nucleofection, before collecting samples for the detection of the SB100X transcript and SB100X protein. To determine the presence, and the splice variant (spliced or unspliced) of the SB100X transcript, multiple RT-PCR reactions were performed. To determine the SB100X protein expression, western blot analysis was performed on the samples. Border RT-PCR reactions were performed, with either both primers flanking the artificial intron, or where one primer was an intron spanning primer. These border PCRs will thus produce a large, amplified product if the transcript is not spliced, or a smaller amplified product if the transcript is spliced. The border RT-PCR with the flanking primers demonstrates correct splicing of intron A (SEQ ID NO: 5), and two splice variants (with the less prominent and smaller band being the correctly spliced transcript) of intron C (SEQ ID NO: 6) in mammalian cells, for both the siSB and siSB-scrambled transposase construct, as can be seen in the left panel of FIG. 5A. The PCR amplified splice variants were also sent for Sanger sequencing, which validated that the lower band was indeed the correctly spliced product. The right panel of FIG. 5A shows the results of another border PCR, using an intron spanning primer, and conditions that would only allow for the amplification of correctly spliced product. FIG. 5B shows expression of Sleeping Beauty transposase protein only in cells transfected with BCMA-siSB-containing intron A but not intron C, in the mammalian HEK293 cells. In contrast, HEK293t cells transfected with BCMA-siSB scrambled show no expression of SB transposase protein. Lastly, the ability of the constructs carrying self-integrating and self-inactivating transposase (siSB) to integrate into the host's (target cells) genome was tested. This was done by keeping a split of the initial transfected cells in culture for an extended period of time and analyzing the expression of BCMA-CAR at multiple timepoints to determine if the expression persisted, compared to the siSB-scrambled constructs that should not show persistence of BCMA-CAR expression. FIG. 5C showed that only the construct carrying BCMA-siSB-intron A can effectively mediate integration of the transposon, leading to longterm persistence of BCMA-CAR expression in mammalian cells; whereas BCMA-siSB-scrambled does not show this persistence in expression due to lack of functional transposase expression (as was corroborated by the western blot analysis). Both constructs, correct and scrambled, harboring intron C do not show persistence of BCMA expression, which would indicate that there is no integration event happening in the host's genome. This corroborates the western blot data, and also the RT-PCR data as the correctly spliced product was minimally produced compared to the other splice variant (this other splice variant would not be able to produce a functional SB protein due to a premature stop codon introduced by the partially spliced artificial intron). To ensure that the AAV siSB vectors can be produced in the insect cell lines, without destroying the vector due to the untimely expression of SB100X, a test was performed to determine whether both introns are spliced (i.e., functional) in the vector producer cells, i.e. insect cells (FIG. 6). To test this, constructs carrying intron A were introduced into insect cells using the Amaxa Cell Line Nucleofector Kit R and the Lonza 2D nucleofector. Nucleofected cells were subsequently examined for presence of the spliced or unspliced transcript using RT-PCR. The data demonstrated (FIG. 6) the presence of only the unspliced SB100X transcript, suggesting that the mammalian intron was not recognized by the insect cells. Such process thus prevented the expression of the transposase protein and the subsequent destruction of the vector genome in the producer cells, which thus allowed production of AAV vectors.

### 3. Example 3: The Disclosed Constructs or Viral Vectors Carrying a Transposon and a Transposase Gene are Functional in Target Cells

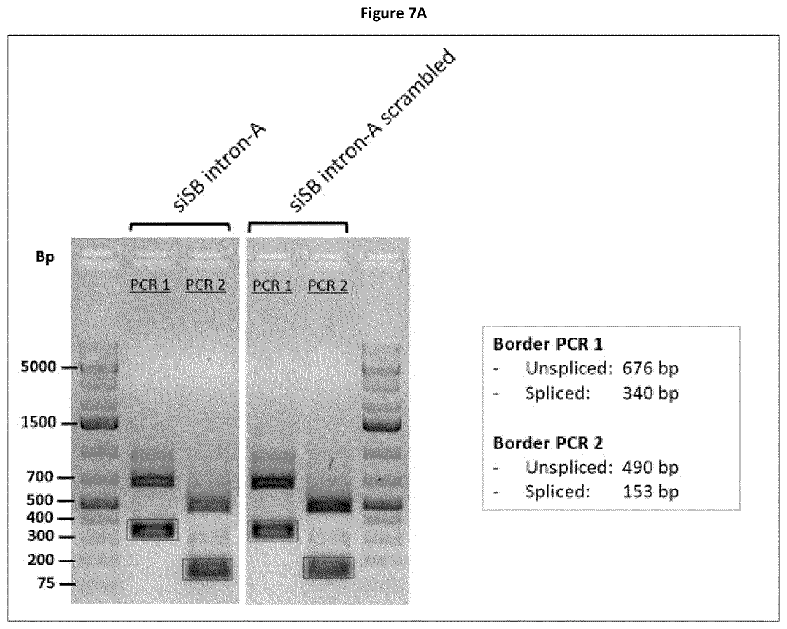

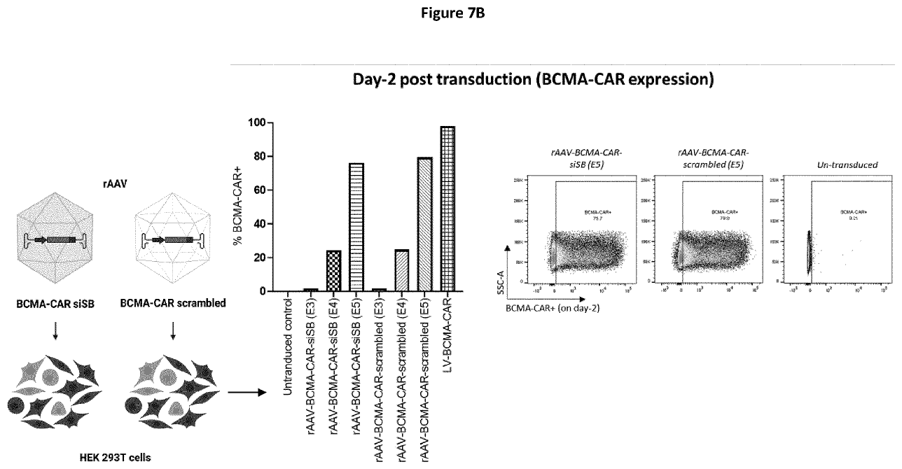

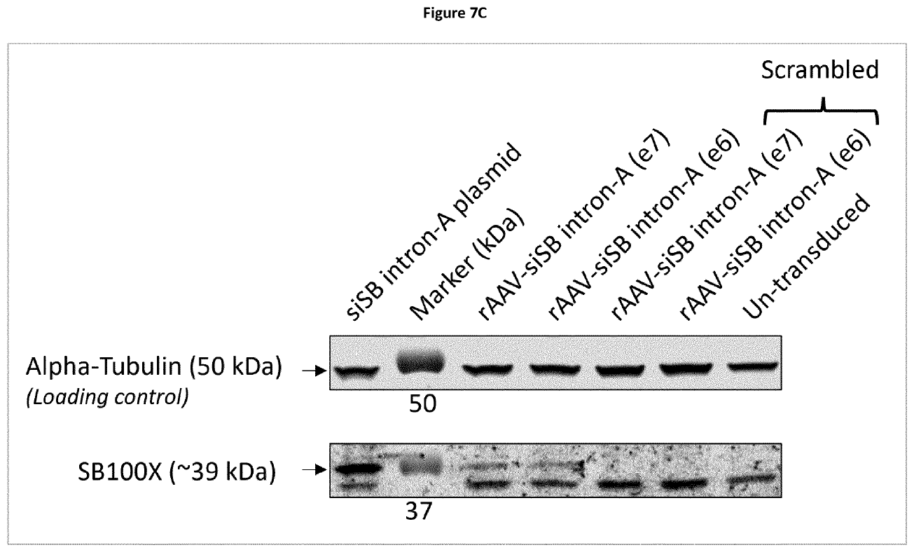

A batch of rAAV viruses (~e12 gc/ml) encoding either BCMA CAR-siSB-A or BCMA CAR-siSB-scrambled-A from a commercial supplier (Virovek) was procured. As quality control, the sizes of the viruses were determined and found to be as expected i.e., 4.7 kb. To determine the functionality of the rAAVs, the rAAVs were tested in HEK293t cells to determine the splicing of the SB transposase mRNA by RT-PCR approaches as described above, to determine the expression of the SB transposase protein by western blotting. To accomplish this, a day before transduction, ~75,000 HEK293t cells were seeded into each well of 24 well plates containing DMEM medium containing serum (10% FBS). The next day, the medium was completely removed from the wells and the cells washed once with DPBS to remove any residual serum. rAAV transductions were performed in transduction-medium (DMEM medium containing 0.5% FBS) at different multiplicity of infections (MOI) of E3, E4, E5. 24 h after transduction, medium was replaced with fresh DMEM medium containing serum (10% FBS). Two days post-transduction, cells were harvested to determine the splicing of SB transposase mRNA, to determine the SB transposase protein production and to determine BCMA CAR expression/stable integration.
a. To determine the splicing of SB transposase mRNA: the border RT-PCRs were performed for the detection of SB100X transcript, as previously mentioned in Example 1. The Border PCRs will amplify a larger product when the SB100X remains unspliced (and thus retains the artificial intron), and a smaller product will be amplified if the transcript is spliced (where the artificial intron is removed, and the SB100X reading frame is restored. FIG. 7A indeed shows the presence of both unspliced as spliced SB100X transcript. As transcription is a continuous process, it was expected to find both forms in the rAAV transfected cells.
b. To determine BCMA CAR expression by flow cytometry: In order detect the BCMA CAR expression on the surface, HEK293t cells transduced with either BCMA CAR-siSB-A or BCMA CAR-siSB-scrambled-A were stained with BCMA CAR detection reagent (CD269 Protein, His & Fc Tag, PE conjugated) and analyzed by flow on a BD LSRFortessa device. A dose dependent correlation between the MOI of rAAV used for transduction and BCMA CAR expression was observed. As shown in FIG. 7B, up to 79% of the population of the cell's expressing BCMA CAR at an MOI of E5 was detected. As a comparison control, HEK293t cells were transduced with lentiviral vector encoding BCMA-CAR.
c. To determine SB transposase protein expression: In order detect the BCMA CAR expression, HEK293t cells transduced with either BCMA CAR-siSB-A or BCMA CAR-siSB-scrambled-A were lysed using RIPA buffer containing protease inhibitors and centrifuged at 10,000 g for 10 min at 4° C. After centrifugation, supernatants were collected and the total protein concentration was estimated using a Micro BCA Protein Assay Kit (ThermoFisher). Approximately 5 μg of the cell lysates were subjected for SDS PAGE by loading the lysates into wells of Bolt 4 to 12%, Bis-Tris, 1.0 mm, Mini Protein Gels (ThermoFisher). The gel was then subjected to western blotting to transfer the proteins from gel onto the PVDF membrane, which was then blocked and incubated with primary antibodies (goat-anti-SB antibody and mouse-alpha-tubulin) followed by incubation with secondary antibodies (donkey-anti-goat antibody conjugated to IRDye 800CW and donkey-anti-mouse antibody conjugated to IRDye 680RD). Finally, the signal was detected using an Odyssey device. As shown in FIG. 7C the expression of Sleeping Beauty transposase was observed only in HEK cells transduced with rAAV encoding BCMA-CAR-siSB but not in HEK cells transduced with BCMA-CAR-siSB scrambled, corroborating the splicing data shown in FIG. 7A and leading to the production of functional protein.

### 4. Example 4: The Disclosed Constructs or Viral Vectors Carrying a Variety of Gene Editing Tools are Functional in Target Cells

A batch of rAAV viruses (~e12 gc/ml) encoding either BCMA CAR-siSB-A or BCMA CAR-siSB. As with siSB, any other genome editing tool such as other transposases, integrases, recombinases, or nucleases are used in similar fashion, where a viral or non-viral delivery vehicle deliver a single fragment of DNA that not only contains the GOI or DNA element that needs to be introduced into the host cell's DNA, but this same fragment of DNA will also contain the enzyme that can incorporate this fragment of DNA in the host cells genome, and self-inactivating itself upon doing so. This can be accomplished when incorporating the DNA recognition motifs of these enzymes in the artificial intron and placing this artificial intron within the enzyme's own coding sequence (CDS). With an integrase such as lambda integrase one would only need to replace the siSB with a self-inactivating Lambda integrase (siLambda), which therefore contain an ATT site (Lambda integrase recognition motif) in the artificial intron located within its own coding sequence. The other ATT site is placed upstream of the GOI cassette, where the left IR is placed in the siSB mechanism (S. Harsha Vijaya Chandra et al., Nucleic Acids Research, Volume 44, Issue 6, 7 Apr. 2016).

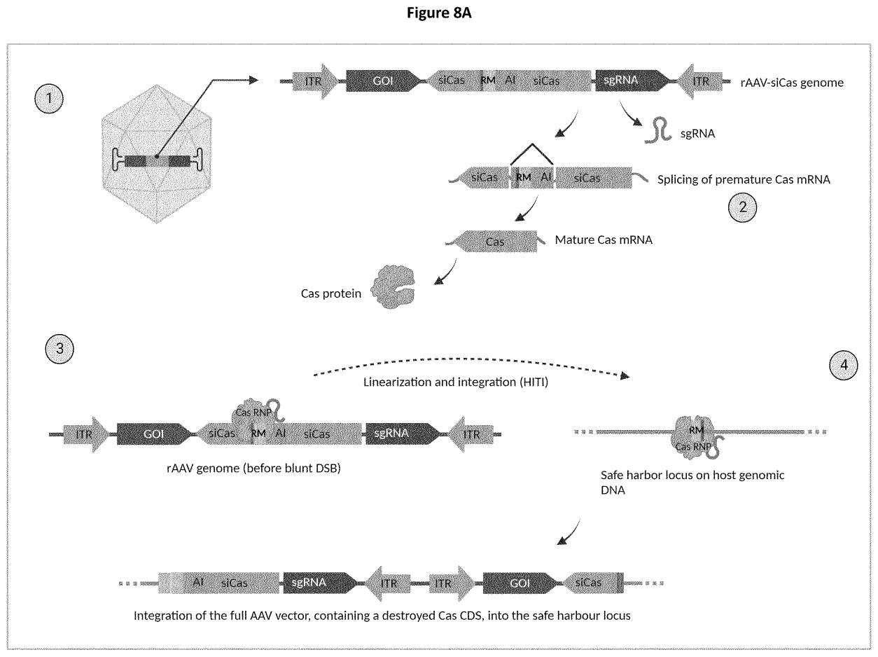

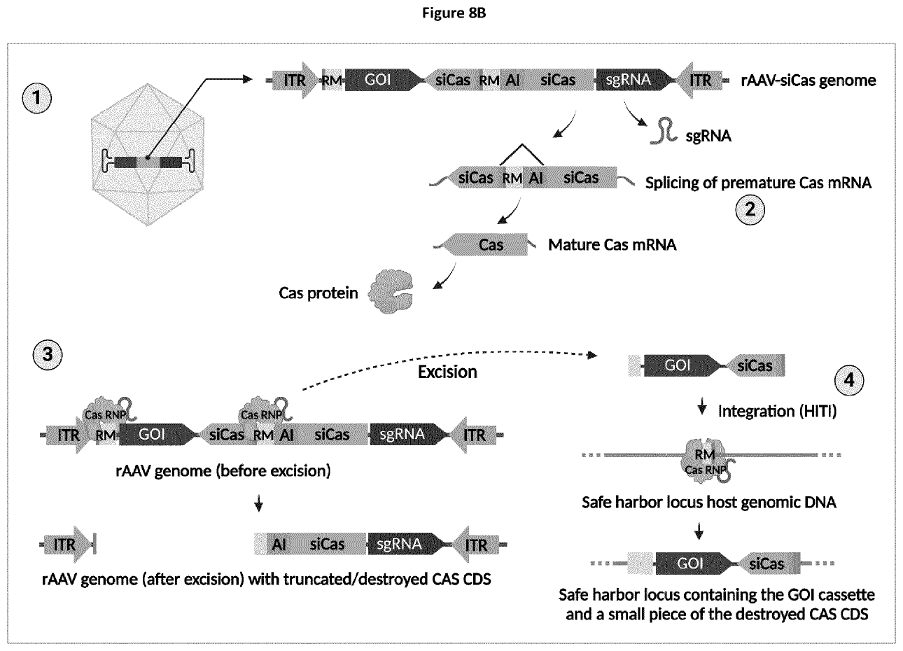

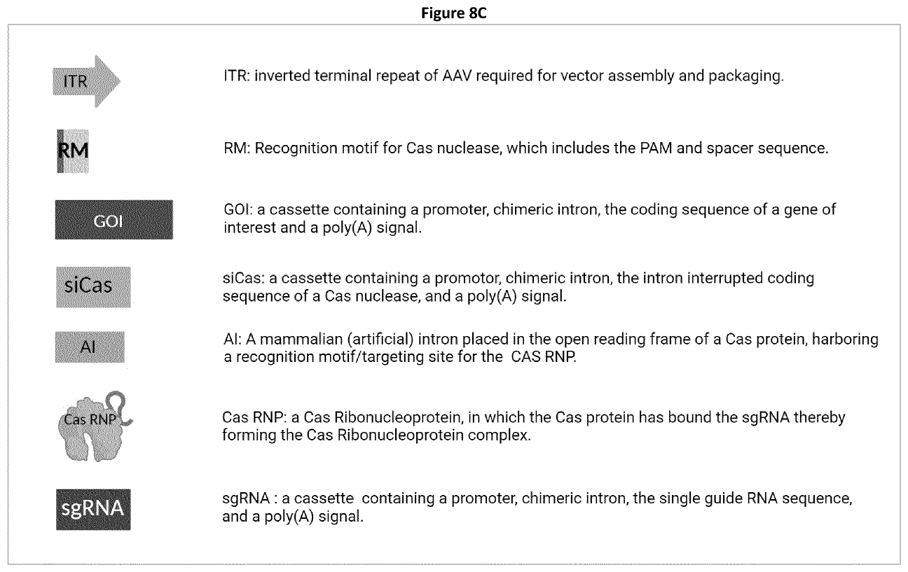

With nucleases, such as but not limited to CRISPR/Cas9 (or any other targeted nuclease that can cause a blunt double stranded break), the recognition motif is incorporated (in this case the PAM and spacer sequence) in the artificial intron. The Cas protein is then initially be produced, as it is spliced out the intron from its own transcript. Upon binding to the sgRNA, the Cas Ribonucleoprotein (RNP) recognizes not only the spacer sequence in the host its genome, but also the identical spacer sequence inside the artificial intron within its own coding sequence. Upon generating the double stranded break (DSB) in the host DNA, it also cuts the template DNA on which the nuclease its own coding sequence is also present, thereby destroying its coding sequence in the process. This DSB in the host its genome is then repaired by Homology Independent Targeted Integration (HITI) where it can incorporate the piece of linearized DNA (S. K. Tsunekawa, et al, Nature 540, 144-149 (2016)). FIG. 8A provides the mechanism when introducing the recognition motif only in the CAS coding sequence (CDS), and FIG. 8B provides the mechanisms when also introducing the recognition motif right upstream of the GOI cassette.

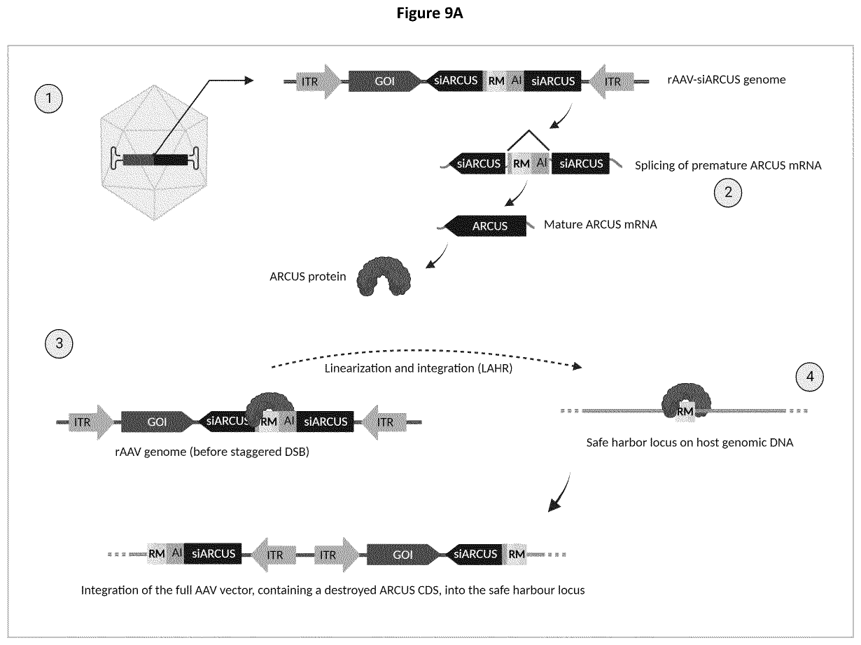

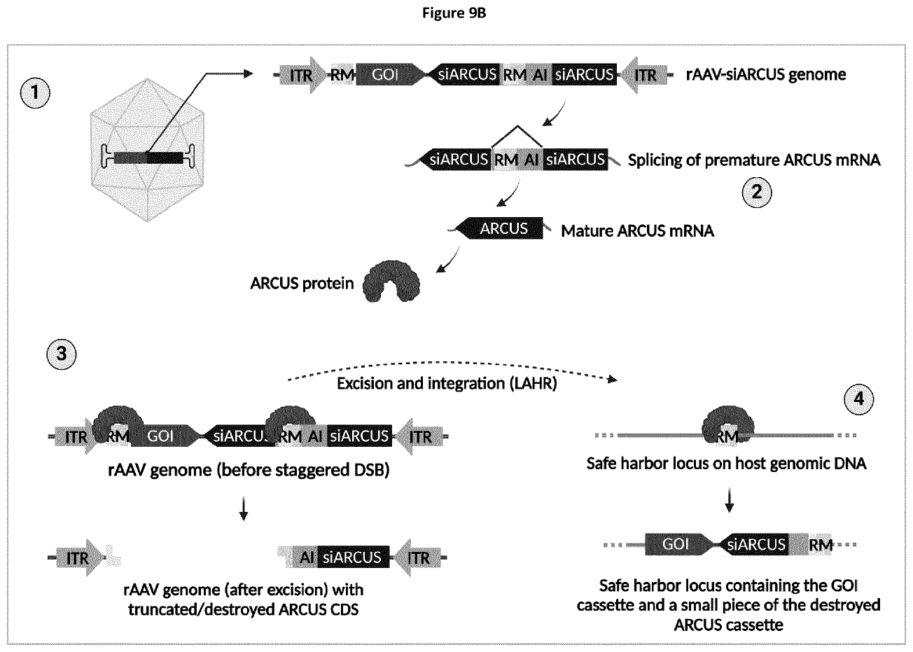

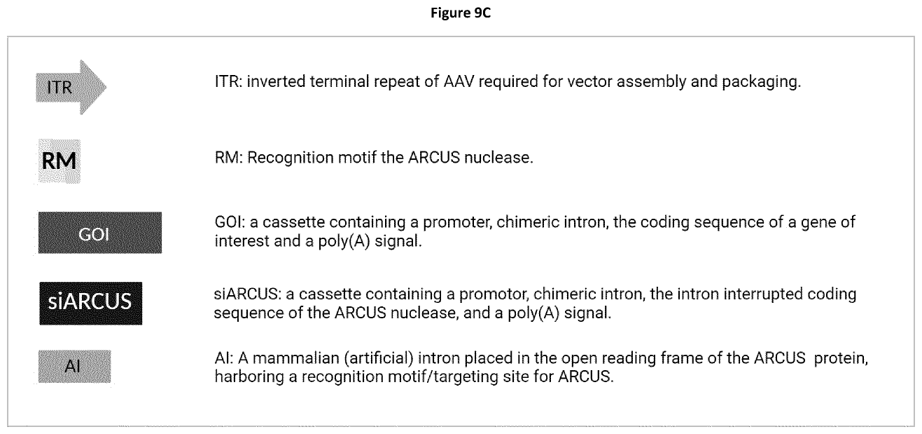

When using nucleases that produce staggered double stranded breaks such as ARCUS nuclease for example, one would again incorporate the recognition motif of ARCUS inside the artificial intron, within the coding sequence of ARCUS itself. This recognition motif is again identical to the recognition motif that it was designed to target in the host cell's genome, thus creating the staggered double stranded break in both the host its genome, as it is in its own CDS within the DNA template that is introduced to the cell to incorporate a specific DNA element(s). When also incorporating short homology (60-80 bp) arms directly adjacent to the staggered DNA end of the DNA template, the double stranded break within the host its genome can be repaired by Ligation-Assisted Homologous Recombination (LAHR) (Zhao Z, et al., Nucleic Acids Res. 2022 Jun. 24; 50(11):e62). FIG. 9A provides a schematic representation of the mechanism when introducing the recognition motif only in the ARCUS CDS, and FIG. 9B provides the mechanisms when introducing the same recognition motif right upstream of the GOI cassette.

In certain embodiments, the viral vector is used to engineer target cells to stably express a GOI for large-scale protein production by transduction of TIE-AAV vector containing a nucleic acid sequence encoding a target protein of interest into host cells.

In certain embodiments, the constructs and viral vectors comprise a protein of interest wherein the protein of interest encompasses, but is not limited to, cytokines, interferons, antibodies, monoclonal antibodies, single-chain variable fragments (scFv), nanobodies, monobodies, insulin, erythropoietin, hormones, food processing enzymes (e.g., amylase, cellulase), enzymes for laundry detergents (e.g., proteases), therapeutic vaccines, clotting factors, growth factors for cell culture, enzymes for biofuel production, and anticoagulants (e.g., tissue plasminogen activator).

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

Many modifications and variations of this disclosure can be made without departing from its spirit and scope, as will be apparent to those skilled in the art. The specific embodiments described herein are offered by way of example only. The embodiments were chosen and described in order to best explain the principles of the disclosure and its practical applications, to thereby enable others skilled in the art to best utilize the disclosure and various embodiments with various modifications as are suited to the particular use contemplated. The disclosure is to be limited only by the terms of the appended claims, along with the full scope of equivalents to which such claims are entitled.

What is claimed is:

1. A polynucleotide comprising (i) a transposable element comprising a pair of inverted repeats, (ii) a first nucleic acid sequence encoding a transposase, and (iii) a mammalian intron positioned the first nucleic acid sequence.

2. The polynucleotide of claim 1, wherein a first inverted repeat in the pair of inverted repeats is positioned upstream of the first nucleic acid sequence and a second inverted repeat in the pair of inverted repeats is positioned within the first nucleic acid sequence.

3. The method of claim 2, wherein the second inverted repeat is positioned within the mammalian intron.

4. The polynucleotide of any one of claims 1-4, wherein the transposable element further comprises a cargo nucleic acid sequence flanked by the pair of inverted repeats.

5. The polynucleotide of claim 4, wherein the cargo nucleic acid sequence encodes an mRNA, a tRNA, an rRNA, an siRNA, a microRNA, a regulating RNA, or a non-coding and coding RNA.

6. The polynucleotide of claim 4 or 5, wherein the cargo nucleic acid sequence comprises a gene of interest.

7. The polynucleotide of any one of claims 4-6, wherein the cargo nucleic acid sequence encodes a therapeutic agent.

8. The polynucleotide of any one of claims 4-7, wherein the cargo nucleic acid sequence does not overlap with the first nucleic acid sequence or the mammalian intron.

9. The polynucleotide of any one of claims 4-8, wherein the transposable element further comprises a transcriptional regulatory element operably linked to the cargo nucleic acid sequence.

10. The polynucleotide of claim 9, wherein the transcriptional regulatory element is a promoter.

11. The polynucleotide of any one of the preceding claims, wherein the transposase is a self-integrating and self-inactivating transposase.

12. The polynucleotide of any one of the preceding claims, wherein the polynucleotide further comprises a pair of AAV inverted terminal repeats flanking the transposable element and the first nucleic acid sequence.

13. The polynucleotide of any one of the preceding claims, wherein when the polynucleotide is within a producer cell, the intron is not spliced thereby making the self-integrating and self-inactivating transposase not functional.

14. The polynucleotide of any one of the preceding claims, wherein when the polynucleotide is within a target cell, the intron is spliced thereby making the self-integrating and self-inactivating transposase functional.

15. The polynucleotide of any one of the preceding claims, wherein when the polynucleotide is within a target cell, the transposase splices the IRs to favor transposition and integration of the cargo in a target region of interest.

16. A polynucleotide comprising (i) first nucleic acid sequence comprising a nucleic acid cassette encoding a Cas nuclease, (ii) a second nucleic acid sequence comprising nucleic acid cassette of a guide RNA (gRNA), and (iii) a mammalian intron positioned the first nucleic acid sequence.

17. The polynucleotide of claim 16, wherein first nucleic acid sequence comprises a nucleic acid sequence of a cargo.

18. The polynucleotide of claim 17, wherein the cargo nucleic acid sequence encodes an mRNA, a tRNA, an rRNA, an siRNA, a microRNA, a regulating RNA, or a non-coding and coding RNA.

19. The polynucleotide of claim 17 or 18, wherein the cargo nucleic acid sequence comprises a gene of interest.

20. The polynucleotide of any one of claims 17-19, wherein the cargo nucleic acid sequence encodes a therapeutic agent.

21. The polynucleotide of any one of claims 17-20, wherein the cargo nucleic acid sequence does not overlap with the first nucleic acid sequence or the mammalian intron.

22. The polynucleotide of any one of claims 16-21, wherein when the polynucleotide is within a producer cell, the intron is not spliced thereby making the Cas nuclease not functional.

23. The polynucleotide of any one of claims 16-22, wherein when the polynucleotide is within a target cell, the Cas nuclease binds to the gRNA thereby favoring gene repair by homology independent targeted integration (HITI) and integration of the cargo.

24. A polynucleotide comprising (i) a nucleic acid sequence comprising a nucleic acid cassette encoding a ARCUS nuclease, and (ii) a mammalian intron positioned the nucleic acid sequence.

25. The polynucleotide of claim 24, wherein the nucleic acid sequence comprises a nucleic acid sequence of a cargo.

26. The polynucleotide of claim 25, wherein the cargo nucleic acid sequence encodes an mRNA, a tRNA, an rRNA, an siRNA, a microRNA, a regulating RNA, or a non-coding and coding RNA.

27. The polynucleotide of claim 25 or 26, wherein the cargo nucleic acid sequence comprises a gene of interest.

28. The polynucleotide of any one of claims 25-27, wherein the cargo nucleic acid sequence encodes a therapeutic agent.

29. The polynucleotide of any one of claims 25-28, wherein the cargo nucleic acid sequence does not overlap with the first nucleic acid sequence or the mammalian intron.

30. The polynucleotide of any one of claims 24-29, wherein when the polynucleotide is within a producer cell, the intron is not spliced thereby making the ARCUS nuclease not functional.

31. The polynucleotide of any one of claims 24-30, wherein when the polynucleotide is within a target cell, the ARCUS nuclease binds to a recognition motif of ARCUS nuclease thereby favoring gene repair by excision, ligation-assisted homologous recombination (LAHR) and integration of the cargo.

32. The polynucleotide of any one of the preceding claims, the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

33. A delivery vehicle comprising the polynucleotide of any one of the claims 1-32.

34. The delivery vehicle of any one of the preceding claims, wherein the delivery vehicle is a lipid nanoparticle (LNP), an extracellular vesicle (EV), an exosome, or a viral vector.

35. The delivery vehicle of any one of the preceding claims, wherein the viral vector comprises an adeno-associated virus (AAV), recombinant adeno-associated virus (rAAV), a parvovirus, a dependovirus, an adenovirus, a lentivirus, or a SV40 virus.

36. The delivery vehicle of any one of the preceding claims, wherein the viral vector is an AAV.

37. The delivery vehicle of any one of the preceding claims, wherein the AAV comprises AAV1, AAV2, AAV3, AAV4, AAV5, AAV6, AAV7, AAV8, or AAV9.

38. The delivery vehicle of any one of the preceding claims, wherein the polynucleotide is flanked by a viral vector inverted terminal repeat (ITR).

39. A cell comprising the polynucleotide of anyone of claims 1-32.

40. A cell comprising the delivery vehicle of any one of claims 33-38.

41. An engineered cell comprising the polynucleotide of anyone of claims 1-32 or the delivery vehicle of any one of claims 33-38.

42. A method of genetically modifying a cell comprising introducing the polynucleotide of anyone of claims 1-32 into the cell.

43. A method of genetically modifying a cell comprising introducing the delivery vehicle of any one of claims 33-38 into the cell.

44. A method of expressing a gene of interest in a cell comprising introducing the polynucleotide of anyone of claims 1-32 into the cell.

45. A method of expressing a gene of interest in a cell comprising introducing the delivery vehicle of any one of claims 33-38 into the cell.

46. The method of claim 44 or claim 45, wherein the cell stably expresses the gene of interest.

47. A method of treating, ameliorating or inhibiting a disease, disorder or condition in a subject in need thereof, the method comprising administering to the subject the polynucleotide of anyone of claims 1-32 or the delivery vehicle of any one of claims 33-38.

48. A method of manufacturing or producing a delivery vehicle in producer cells, wherein the delivery vehicle comprises the polynucleotide of any one of the claims 1-32.

49. A method of manufacturing or producing a cell comprising a delivery vehicle comprising the polynucleotide of any one of the claims 1-32.

50. A method for integrating a cargo nucleic acid sequence into the genome of a mammalian cell, the method comprising introducing in the mammalian cell a polynucleotide comprising (i) a transposable element comprising the cargo nucleic acid sequence and a pair of inverted repeats, (ii) a first nucleic acid sequence encoding a transposase, and (iii) a mammalian intron positioned within the first nucleic acid sequence.

51. The method of claim 50, wherein the transposase is activated upon the splicing of the intron.

52. The method claim 51, wherein the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

53. The method of any one of claims 50-52, wherein the activated transposase splices the IR to favor transposition and integration of the gene of interest in a target region of interest.

54. A method for integrating a cargo nucleic acid sequence into the genome of a mammalian cell, the method comprising introducing in the mammalian cell a polynucleotide comprising (i) a first nucleic acid sequence comprising a nucleic acid cassette encoding a Cas nuclease, (ii) a second nucleic acid sequence comprising nucleic acid cassette of a guide RNA (gRNA), and (iii) a mammalian intron positioned the first nucleic acid sequence.

55. The method claim 54, wherein the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

56. The method of any one of claims 54-55, wherein the Cas nuclease is activated upon splicing of the intron.

57. The method of claim 56, wherein the activated Cas nuclease binds to the gRNA thereby favoring gene repair by homology independent targeted integration (HITI) and integration of the gene of interest.

58. A method for integrating a cargo nucleic acid sequence into the genome of a mammalian cell, the method comprising introducing in the mammalian cell a polynucleotide comprising (i) a nucleic acid sequence comprising a nucleic acid cassette encoding a ARCUS nuclease, and (ii) a mammalian intron positioned the nucleic acid sequence.

59. The method claim 58, wherein the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

60. The method of any one of claims 58-59, wherein the ARCUS nuclease is activated upon splicing of the intron.

61. The method of claim 60, wherein the activated ARCUS nuclease binds to a recognition motif of ARCUS nuclease thereby favoring gene repair by excision, ligation-assisted homologous recombination (LAHR) and integration of the gene of interest.

62. A construct for delivering a nucleic acid into a cell genome comprising:
a) a first cassette comprising:
i) an inverted repeat of a transposon;
ii) an enhancer;
iii) a promoter;
iv) an intron;
v) a target antigen coding sequence; and
vi) a poly(A) signal; and
b) a second cassette comprising:
i) a promoter;
ii) a first intron;
iii) a first amino acid coding sequence of a transposase;
iii) a first part of a second intron;
iv) a transposase specific inverted repeat;
v) a second part of the second intron;
vi) a second amino acid coding sequence of the transpose; and
vii) a poly(A) signal,
wherein the second cassette is functional in a target cell and not functional in a producer cell.

63. A delivery vehicle for delivering a nucleic acid into a cell genome comprising:
a) a first cassette comprising:
i) an inverted repeat of a transposon;
ii) an enhancer;
iii) a promoter;
iv) an intron;
v) a target antigen coding sequence; and
vi) a poly(A) signal; and
b) a second cassette comprising:
i) a promoter;
ii) a first intron;
iii) a first amino acid coding sequence of a transposase;
iii) a first part of a second intron;
iv) a transposase specific inverted repeat;
v) a second part of the second intron;
vi) a second amino acid coding sequence of the transpose; and
vii) a poly(A) signal,
wherein the second cassette is functional in a target cell and not functional in a producer cell.

64. A viral vector for delivery of nucleic acid into a cell genome comprising:
a) a first nucleic acid encoding a transposon comprising a gene of interest; and
b) a second nucleic acid encoding a transposase that is functional in target cells and not functional in producer cells.

65. The viral vector of claim 64, wherein the second nucleic acid encodes a transposase coding region which is interrupted with an intron that is spliced in target cells and not spliced in producer cells.

66. The viral vector of claim 65, wherein the intron comprises a sequence homology of at least 85%, 86%, 87%, 88%, 89%, 90%, 91%, 92%, 93%, 94%, 95%, 96%, 97%, 98%, 99% or 100% to SEQ ID NO: 5, SEQ ID NO: 6, and SEQ ID NO: 13.

67. A cell comprising a viral vector of any one of claims 64-66.

68. A method of genetically modifying a cell comprising introducing a viral vector of any one of claims 64-66 into a cell.

69. A method of gene transfer using the viral vector of any one of claims 64-66.

70. A method of expressing a gene of interest in a cell comprising introducing the viral vector of any one of claims 64-66 into a cell.

71. The method of claim 70, wherein the cell stably expresses the gene of interest.

72. An engineer cell comprising a viral vector of any one of claims 64-66.

73. A method of treating, ameliorating or inhibiting a disease, disorder or condition with a viral vector of any one of claims 64-66.

74. A method of manufacturing or producing a viral vector in producer cells, wherein the vector comprises:
a) a first nucleic acid encoding a transposon comprising a gene of interest; and
b) a second nucleic acid encoding a transposase that is functional in target cells and not functional in producer cells.

75. A method of claim 74, wherein the vector comprises an intron and wherein the producer cells are unable to splice the intron.

76. A method of manufacturing or producing a cell comprising a viral vector, wherein the vector comprises:
a) a first nucleic acid encoding a transposon comprising a gene of interest; and
b) a second nucleic acid encoding a transposase that is functional in target cells and not functional in producer cells.

77. A construct for delivery of nucleic acid into a cell genome comprising:
a) a first nucleic acid encoding a transposon comprising a gene of interest; and
b) a second nucleic acid encoding a transposase that is functional in target cells and not functional in producer cells.

78. The construct of claim 77, wherein the second nucleic acid encoding a transposase further comprises an intron that splices in target cells and does not splice in producer cells.

79. A construct for delivery of nucleic acid into a cell genome comprising:
a) a first cassette comprising:
i) an inverted repeat of a transposon;
ii) an enhancer;
iii) a promoter;
iv) an intron;
v) a target antigen coding sequence; and
vi) a poly(A) signal; and
b) a second cassette comprising:
i) a promoter;
ii) a first intron;
iii) a first amino acid coding sequence of a transposase;
iii) a first part of a second intron;
iv) a transposase specific inverted repeat;
v) a second part of the second intron;
vi) a second amino acid coding sequence of the transpose; and
vii) a poly(A) signal,
wherein the second cassette is functional in target cells and not functional in producer cells.

80. A viral vector for delivery of nucleic acid into a cell genome comprising:
a) a first cassette comprising:
i) an inverted repeat of a transposon;
ii) an enhancer;
iii) a promoter;
iv) an intron;
v) a target antigen coding sequence; and
vi) a poly(A) signal; and
b) a second cassette comprising:
i) a promoter;
ii) a first intron;
iii) a first amino acid coding sequence of a transposase;
iii) a first part of a second intron;
iv) a transposase specific inverted repeat;
v) a second part of the second intron;
vi) a second amino acid coding sequence of the transpose; and
vii) a poly(A) signal,
wherein the second cassette is functional in target cells and not functional in producer cells.


SEQUENCE LISTING (14 sequences)

SEQ ID NO: 1 - DNA, 4651 bp, synthetic construct
  misc_feature 1..4651: note = Artificially synthesized
  source 1..4651: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctaatatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagactagtgcgcgcggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacagggcgcgccaccatggccctccccgtgacagccctgctgttgcctctggctctgctgctgcacgccgctagacccgaagtgcagctgctggaaagcggcggcggactggttcagcctggcggaagcctgcggctgtcttgtgccgccagcggcttcacttttagcagctacgccatgagctgggtgaggcaggcccctggaaagggcctggaatgggtctccagcatctctggcagcggcgactacatctactacgccgacagcgtgaaaggcagattcaccatcagcagagacatctcaaagaacaccctgtatctgcagatgaactccctgagagccgaagataccgccgtgtactactgcgctaaggaaggcaccggcgccaacagcagcctcgctgattacagaggccagggaaccctggtcaccgtgtccagcttcgtgcccgtgttcctgcccgctaagcctaccaccacacctgctccaagacctccaacacccgcgcctacaattgcctctcagccactgagcctgagacctgaggcctgcagacccgccgccggcggcgctgtgcacaccagaggcctggatttcgcctgtgatatctacatctgggcccccctggccggaacatgcggcgtgctgctgctgtctctggtgatcaccctgtactgcaaccacagaaacaagagaggcagaaagaagctgctgtacattttcaagcagcctttcatgagaccagtgcagaccacccaggaggaagatggctgtagctgtagattccccgaggaagaagagggcggctgcgagctgcgggtgaagttcagccgctccgccgacgcccctgcctatcaacagggccagaaccagctgtacaacgagctgaacctgggcagaagagaggaatacgacgtgctggacaagagacggggaagagatcctgagatgggcggaaaaccgcggcggaaaaatccacaggagggcctgtacaatgagctgcaaaaggacaagatggccgaggcctacagcgagatcggcatgaagggcgagagaagacgtggcaagggccacgacggcctgtaccagggcctgtctacagccacaaaagacacctacgatgcattgcatatgcaggccctgcctcccagatgacctgcaggtggccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatctagcttgtggaaggctactcgaaatggcatgcgaattcaacttgtttattgcagcttataatggttacaaataaagcaatagcatcacaaatttcacaaataaagcatttttttcactgcattctagttgtggtttgtccaaactcatcaatgtatcttatcatgtctggatccccgcggttagggcccctagtatttggtagcattgcctttaaattgtttaacttgggtcaaacgtttcgggtagccttccacaagcttcccacaataatttgggtgaattttggcccattcctcctgacagagctggtgtaactgagtcaggtttgtaggcctccttgctcgcacacgctttttcagttctgcccacaaattttctataggattgaggtcagggctttgtgatggccactccaataccttgactttgttgtccttaagccattttgccacaactttggaagtatgcttggggtcattgtcgtgttggaagacccatttgcgaccaagctttaacttcctgactgatgtcttgagatgttgcttcaatatatccacatactgcaccgcgtccatgatgccatctattttgtgaagtgcaccagtccctcctgcagcaaagcacccccacaacatgatgctgccacccccgtgcttcacggttgggatggtgttcttcggcttgcaagcctcccccttcttcctccaaacataacgatggtcattatggccaaacagttctatttttgtttcatcagaccagaggacatttctccaaaaagtacgatctttgtccccatgtgcagttgcaaaccgtagtctggctttcttatgtcggttttggagcagtggcttcttccttgctgagtggcctttcaggttatgtcgatataggactcgttttactgtggatatagatacttttgtacctgtttcctaggaaagaggcaaggaaaggtgataaaagtgaatctgaggcatactcgagttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatacgtacgactgcacccttggtctcctccaccgcttcttgtcctgcttgcttacctccagcatcttcacaaggtcctttgctgttgttctgggattgatttgcacttttcgcaccaaagtacgttcatctctaggagacagaacgcgtctccttcctgagcggtatgacggctgcgtggtcccatggtgtttatacttgcgtactattgtttgtacagatgaacgtggtaccgccaggcgtttggaaattgctcccaaggatgaaccagacttgtggaggtctacaattctttttctgaggtcttggctgatttcttttgattttcccatggtggcctagaagtaaaggcaacatccactgaggagcagttctttgatttgcaccaccaccggatccgggacctgaaataaaagacaaaaagactaaacttactttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 4817 bp, synthetic construct
  misc_feature 1..4817: note = Artificially synthesized
  source 1..4817: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctaatatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagactagtgcgcgcggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacagggcgcgccaccatggccctccccgtgacagccctgctgttgcctctggctctgctgctgcacgccgctagacccgaagtgcagctgctggaaagcggcggcggactggttcagcctggcggaagcctgcggctgtcttgtgccgccagcggcttcacttttagcagctacgccatgagctgggtgaggcaggcccctggaaagggcctggaatgggtctccagcatctctggcagcggcgactacatctactacgccgacagcgtgaaaggcagattcaccatcagcagagacatctcaaagaacaccctgtatctgcagatgaactccctgagagccgaagataccgccgtgtactactgcgctaaggaaggcaccggcgccaacagcagcctcgctgattacagaggccagggaaccctggtcaccgtgtccagcttcgtgcccgtgttcctgcccgctaagcctaccaccacacctgctccaagacctccaacacccgcgcctacaattgcctctcagccactgagcctgagacctgaggcctgcagacccgccgccggcggcgctgtgcacaccagaggcctggatttcgcctgtgatatctacatctgggcccccctggccggaacatgcggcgtgctgctgctgtctctggtgatcaccctgtactgcaaccacagaaacaagagaggcagaaagaagctgctgtacattttcaagcagcctttcatgagaccagtgcagaccacccaggaggaagatggctgtagctgtagattccccgaggaagaagagggcggctgcgagctgcgggtgaagttcagccgctccgccgacgcccctgcctatcaacagggccagaaccagctgtacaacgagctgaacctgggcagaagagaggaatacgacgtgctggacaagagacggggaagagatcctgagatgggcggaaaaccgcggcggaaaaatccacaggagggcctgtacaatgagctgcaaaaggacaagatggccgaggcctacagcgagatcggcatgaagggcgagagaagacgtggcaagggccacgacggcctgtaccagggcctgtctacagccacaaaagacacctacgatgcattgcatatgcaggccctgcctcccagatgacctgcaggtggccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatctagcttgtggaaggctactcgaaatggcatgcgaattcaacttgtttattgcagcttataatggttacaaataaagcaatagcatcacaaatttcacaaataaagcatttttttcactgcattctagttgtggtttgtccaaactcatcaatgtatcttatcatgtctggatccccgcggttagggcccctagtatttggtagcattgcctttaaattgtttaacttgggtcaaacgtttcgggtagccttccacaagcttcccacaataatttgggtgaattttggcccattcctcctgacagagctggtgtaactgagtcaggtttgtaggcctccttgctcgcacacgctttttcagttctgcccacaaattttctataggattgaggtcagggctttgtgatggccactccaataccttgactttgttgtccttaagccattttgccacaactttggaagtatgcttggggtcattgtcgtgttggaagacccatttgcgaccaagctttaacttcctgactgatgtcttgagatgttgcttcaatatatccacatactgcaccgcgtccatgatgccatctattttgtgaagtgcaccagtccctcctgcagcaaagcacccccacaacatgatgctgccacccccgtgcttcacggttgggatggtgttcttcggcttgcaagcctcccccttcttcctccaaacataacgatggtcattatggccaaacagttctatttttgtttcatcagaccagaggacatttctccaaaaagtacgatctttgtccccatgtgcagttgcaaaccgtagtctggctttcttatgtcggttttggagcagtggcttcttccttgctgagtggcctttcaggttatgtcgatataggactcgttttactgtggatatagatacttttgtacctgtttcctggggagaaaccagagggcaacagagggagccggagagcaagaggccagcatctccctgctccaggagctgtttgtttttctctctccatcctccagggagccaggagctttctgagattggccaaatctcgagttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatacgtacgactgggcttacatggcgatactcacattcagaaccccaaaccctgagggttagtgcccccgtcccatctacaggtcgctgcctctcccctcaggacacattgtgccaaagggattttaggggcgcttacctccagcatcttcacaaggtcctttgctgttgttctgggattgatttgcacttttcgcaccaaagtacgttcatctctaggagacagaacgcgtctccttcctgagcggtatgacggctgcgtggtcccatggtgtttatacttgcgtactattgtttgtacagatgaacgtggtaccgccaggcgtttggaaattgctcccaaggatgaaccagacttgtggaggtctacaattctttttctgaggtcttggctgatttcttttgattttcccatggtggcctagaagtaaaggcaacatccactgaggagcagttctttgatttgcaccaccaccggatccgggacctgaaataaaagacaaaaagactaaacttactttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 4651 bp, synthetic construct
  misc_feature 1..4651: note = Artificially synthesized
  source 1..4651: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctaatatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagactagtgcgcgcggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacagggcgcgccaccatggccctccccgtgacagccctgctgttgcctctggctctgctgctgcacgccgctagacccgaagtgcagctgctggaaagcggcggcggactggttcagcctggcggaagcctgcggctgtcttgtgccgccagcggcttcacttttagcagctacgccatgagctgggtgaggcaggcccctggaaagggcctggaatgggtctccagcatctctggcagcggcgactacatctactacgccgacagcgtgaaaggcagattcaccatcagcagagacatctcaaagaacaccctgtatctgcagatgaactccctgagagccgaagataccgccgtgtactactgcgctaaggaaggcaccggcgccaacagcagcctcgctgattacagaggccagggaaccctggtcaccgtgtccagcttcgtgcccgtgttcctgcccgctaagcctaccaccacacctgctccaagacctccaacacccgcgcctacaattgcctctcagccactgagcctgagacctgaggcctgcagacccgccgccggcggcgctgtgcacaccagaggcctggatttcgcctgtgatatctacatctgggcccccctggccggaacatgcggcgtgctgctgctgtctctggtgatcaccctgtactgcaaccacagaaacaagagaggcagaaagaagctgctgtacattttcaagcagcctttcatgagaccagtgcagaccacccaggaggaagatggctgtagctgtagattccccgaggaagaagagggcggctgcgagctgcgggtgaagttcagccgctccgccgacgcccctgcctatcaacagggccagaaccagctgtacaacgagctgaacctgggcagaagagaggaatacgacgtgctggacaagagacggggaagagatcctgagatgggcggaaaaccgcggcggaaaaatccacaggagggcctgtacaatgagctgcaaaaggacaagatggccgaggcctacagcgagatcggcatgaagggcgagagaagacgtggcaagggccacgacggcctgtaccagggcctgtctacagccacaaaagacacctacgatgcattgcatatgcaggccctgcctcccagatgacctgcaggtggccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatctagcttgtggaaggctactcgaaatggcatgcgaattcaacttgtttattgcagcttataatggttacaaataaagcaatagcatcacaaatttcacaaataaagcatttttttcactgcattctagttgtggtttgtccaaactcatcaatgtatcttatcatgtctggatccccgcggttagggcccctagtatttggtagcattgcctttaaattgtttaacttgggtcaaacgtttcgggtagccttccacaagcttcccacaataatttgggtgaattttggcccattcctcctgacagagctggtgtaactgagtcaggtttgtaggcctccttgctcgcacacgctttttcagttctgcccacaaattttctataggattgaggtcagggctttgtgatggccactccaataccttgactttgttgtccttaagccattttgccacaactttggaagtatgcttggggtcattgtcgtgttggaagacccatttgcgaccaagctttaacttcctgactgatgtcttgagatgttgcttcaatatatccacatactgcaccgcgtccatgatgccatctattttgtgaagtgcaccagtccctcctgcagcaaagcacccccacaacatgatgctgccacccccgtgcttcacggttgggatggtgttcttcggcttgcaagcctcccccttcttcctccaaacataacgatggtcattatggccaaacagttctatttttgtttcatcagaccagaggacatttctccaaaaagtacgatctttgtccccatgtgcagttgcaaaccgtagtctggctttcttatgtcggttttggagcagtggcttcttccttgctgagtggcctttcaggttatgtcgatataggactcgttttactgtggatatagatacttttgtacctgtttcctaggaaagaggcaaggaaaggtgataaaagtgaatctgaggcatactcgagttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatacgtacgactgcacccttggtctcctccaccgcttcttgtcctgcttgcttacctccagcatcttcacaaggtcctttgctgttgttctgggattgatttgcacttttcgcaccaaagtacgttcatctctaggagacagaacgcgtctccttccttagcgctatgacggctgcgtggtcccatggtgtttatacttgcgtactattgtttgtacagatgaacgtggtaccgccaggcgtttggaaattgctcccaaggatgaaccagacttgtggaggtctacaattctttttctgaggtcttggctgatttcttttgattttcccatggtggcctagaagtaaaggcaacatccactgaggagcagttctttgatttgcaccaccaccggatccgggacctgaaataaaagacaaaaagactaaacttactttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 4817 bp, synthetic construct
  misc_feature 1..4817: note = Artificially synthesized
  source 1..4817: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctaatatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagactagtgcgcgcggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacagggcgcgccaccatggccctccccgtgacagccctgctgttgcctctggctctgctgctgcacgccgctagacccgaagtgcagctgctggaaagcggcggcggactggttcagcctggcggaagcctgcggctgtcttgtgccgccagcggcttcacttttagcagctacgccatgagctgggtgaggcaggcccctggaaagggcctggaatgggtctccagcatctctggcagcggcgactacatctactacgccgacagcgtgaaaggcagattcaccatcagcagagacatctcaaagaacaccctgtatctgcagatgaactccctgagagccgaagataccgccgtgtactactgcgctaaggaaggcaccggcgccaacagcagcctcgctgattacagaggccagggaaccctggtcaccgtgtccagcttcgtgcccgtgttcctgcccgctaagcctaccaccacacctgctccaagacctccaacacccgcgcctacaattgcctctcagccactgagcctgagacctgaggcctgcagacccgccgccggcggcgctgtgcacaccagaggcctggatttcgcctgtgatatctacatctgggcccccctggccggaacatgcggcgtgctgctgctgtctctggtgatcaccctgtactgcaaccacagaaacaagagaggcagaaagaagctgctgtacattttcaagcagcctttcatgagaccagtgcagaccacccaggaggaagatggctgtagctgtagattccccgaggaagaagagggcggctgcgagctgcgggtgaagttcagccgctccgccgacgcccctgcctatcaacagggccagaaccagctgtacaacgagctgaacctgggcagaagagaggaatacgacgtgctggacaagagacggggaagagatcctgagatgggcggaaaaccgcggcggaaaaatccacaggagggcctgtacaatgagctgcaaaaggacaagatggccgaggcctacagcgagatcggcatgaagggcgagagaagacgtggcaagggccacgacggcctgtaccagggcctgtctacagccacaaaagacacctacgatgcattgcatatgcaggccctgcctcccagatgacctgcaggtggccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggatctagcttgtggaaggctactcgaaatggcatgcgaattcaacttgtttattgcagcttataatggttacaaataaagcaatagcatcacaaatttcacaaataaagcatttttttcactgcattctagttgtggtttgtccaaactcatcaatgtatcttatcatgtctggatccccgcggttagggcccctagtatttggtagcattgcctttaaattgtttaacttgggtcaaacgtttcgggtagccttccacaagcttcccacaataatttgggtgaattttggcccattcctcctgacagagctggtgtaactgagtcaggtttgtaggcctccttgctcgcacacgctttttcagttctgcccacaaattttctataggattgaggtcagggctttgtgatggccactccaataccttgactttgttgtccttaagccattttgccacaactttggaagtatgcttggggtcattgtcgtgttggaagacccatttgcgaccaagctttaacttcctgactgatgtcttgagatgttgcttcaatatatccacatactgcaccgcgtccatgatgccatctattttgtgaagtgcaccagtccctcctgcagcaaagcacccccacaacatgatgctgccacccccgtgcttcacggttgggatggtgttcttcggcttgcaagcctcccccttcttcctccaaacataacgatggtcattatggccaaacagttctatttttgtttcatcagaccagaggacatttctccaaaaagtacgatctttgtccccatgtgcagttgcaaaccgtagtctggctttcttatgtcggttttggagcagtggcttcttccttgctgagtggcctttcaggttatgtcgatataggactcgttttactgtggatatagatacttttgtacctgtttcctggggagaaaccagagggcaacagagggagccggagagcaagaggccagcatctccctgctccaggagctgtttgtttttctctctccatcctccagggagccaggagctttctgagattggccaaatctcgagttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatacgtacgactgggcttacatggcgatactcacattcagaaccccaaaccctgagggttagtgcccccgtcccatctacaggtcgctgcctctcccctcaggacacattgtgccaaagggattttaggggcgcttacctccagcatcttcacaaggtcctttgctgttgttctgggattgatttgcacttttcgcaccaaagtacgttcatctctaggagacagaacgcgtctccttccttagcgctatgacggctgcgtggtcccatggtgtttatacttgcgtactattgtttgtacagatgaacgtggtaccgccaggcgtttggaaattgctcccaaggatgaaccagacttgtggaggtctacaattctttttctgaggtcttggctgatttcttttgattttcccatggtggcctagaagtaaaggcaacatccactgaggagcagttctttgatttgcaccaccaccggatccgggacctgaaataaaagacaaaaagactaaacttactttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 92 bp, synthetic construct
  misc_feature 1..92: note = Artificially synthesized
  source 1..92: mol_type = other DNA
gtaagcaagcaggacaagaagcggtggaggagaccaagggtgcagttatgcctcagattcacttttatcacctttccttgcctctttcctag

SEQ ID NO: 6 - DNA, 258 bp, synthetic construct
  misc_feature 1..258: note = Artificially synthesized
  source 1..258: mol_type = other DNA
gtaagcgcccctaaaatccctttggcacaatgtgtcctgaggggagaggcagcgacctgtagatgggacgggggcactaaccctcagggtttggggttctgaatgtgagtatcgccatgtaagcccagtatttggccaatctcagaaagctcctggctccctggaggatggagagagaaaaacaaacagctcctggagcagggagatgctggcctcttgctctccggctccctctgttgccctctggtttctccccag

SEQ ID NO: 7 - DNA, 1359 bp, synthetic construct
  misc_feature 1..1359: note = Artificially synthesized
  source 1..1359: mol_type = other DNA
atgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggtaagcaagcaggacaagaagcggtggaggagaccaagggtgcagtcgtacgtatacagttgaagtcggaagtttacatacaccttagccaaatacatttaaactcactttttcacaattcctgacatttaatcctagtaaaaattccctgtcttaggtcagttaggatcaccactttattttaagaatgtgaaatatcagaataatagtagagagaatgattcatttcagcttttatttctttcatcacattcccagtgggtcagaagtttacatacactcaactcgagtatgcctcagattcacttttatcacctttccttgcctctttcctaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggtcttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactag

SEQ ID NO: 8 - DNA, 807 bp, synthetic construct
  misc_feature 1..807: note = Artificially synthesized
  source 1..807: mol_type = other DNA
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacag

SEQ ID NO: 9 - DNA, 1140 bp, synthetic construct
  misc_feature 1..1140: note = Artificially synthesized
  source 1..1140: mol_type = other DNA
atggccctccccgtgacagccctgctgttgcctctggctctgctgctgcacgccgctagacccgaagtgcagctgctggaaagcggcggcggactggttcagcctggcggaagcctgcggctgtcttgtgccgccagcggcttcacttttagcagctacgccatgagctgggtgaggcaggcccctggaaagggcctggaatgggtctccagcatctctggcagcggcgactacatctactacgccgacagcgtgaaaggcagattcaccatcagcagagacatctcaaagaacaccctgtatctgcagatgaactccctgagagccgaagataccgccgtgtactactgcgctaaggaaggcaccggcgccaacagcagcctcgctgattacagaggccagggaaccctggtcaccgtgtccagcttcgtgcccgtgttcctgcccgctaagcctaccaccacacctgctccaagacctccaacacccgcgcctacaattgcctctcagccactgagcctgagacctgaggcctgcagacccgccgccggcggcgctgtgcacaccagaggcctggatttcgcctgtgatatctacatctgggcccccctggccggaacatgcggcgtgctgctgctgtctctggtgatcaccctgtactgcaaccacagaaacaagagaggcagaaagaagctgctgtacattttcaagcagcctttcatgagaccagtgcagaccacccaggaggaagatggctgtagctgtagattccccgaggaagaagagggcggctgcgagctgcgggtgaagttcagccgctccgccgacgcccctgcctatcaacagggccagaaccagctgtacaacgagctgaacctgggcagaagagaggaatacgacgtgctggacaagagacggggaagagatcctgagatgggcggaaaaccgcggcggaaaaatccacaggagggcctgtacaatgagctgcaaaaggacaagatggccgaggcctacagcgagatcggcatgaagggcgagagaagacgtggcaagggccacgacggcctgtaccagggcctgtctacagccacaaaagacacctacgatgcattgcatatgcaggccctgcctcccagatga

SEQ ID NO: 10 - DNA, 293 bp, synthetic construct
  misc_feature 1..293: note = Artificially synthesized
  source 1..293: mol_type = other DNA
tagaagtaaaggcaacatccactgaggagcagttctttgatttgcaccaccaccggatccgggacctgaaataaaagacaaaaagactaaacttactttgcaaaagcctaggcctccaaaaaagcctcctcactacttctggaatagctcagaggccgaggcggcctcggcctctgcataaataaaaaaaattagtcagcgatggggcggagaatgggcggaactgggcggagttaggggcgggatgggcggagttaggggcgggactatggttgctgactaattgagatgca

SEQ ID NO: 11 - DNA, 1023 bp, synthetic construct
  misc_feature 1..1023: note = Artificially synthesized
  source 1..1023: mol_type = other DNA
tagtatttggtagcattgcctttaaattgtttaacttgggtcaaacgtttcgggtagccttccacaagcttcccacaataatttgggtgaattttggcccattcctcctgacagagctggtgtaactgagtcaggtttgtaggcctccttgctcgcacacgctttttcagttctgcccacaaattttctataggattgaggtcagggctttgtgatggccactccaataccttgactttgttgtccttaagccattttgccacaactttggaagtatgcttggggtcattgtcgtgttggaagacccatttgcgaccaagctttaacttcctgactgatgtcttgagatgttgcttcaatatatccacatactgcaccgcgtccatgatgccatctattttgtgaagtgcaccagtccctcctgcagcaaagcacccccacaacatgatgctgccacccccgtgcttcacggttgggatggtgttcttcggcttgcaagcctcccccttcttcctccaaacataacgatggtcattatggccaaacagttctatttttgtttcatcagaccagaggacatttctccaaaaagtacgatctttgtccccatgtgcagttgcaaaccgtagtctggctttcttatgtcggttttggagcagtggcttcttccttgctgagtggcctttcaggttatgtcgatataggactcgttttactgtggatatagatacttttgtacctgtttcctccagcatcttcacaaggtcctttgctgttgttctgggattgatttgcacttttcgcaccaaagtacgttcatctctaggagacagaacgcgtctccttcctgagcggtatgacggctgcgtggtcccatggtgtttatacttgcgtactattgtttgtacagatgaacgtggtaccgccaggcgtttggaaattgctcccaaggatgaaccagacttgtggaggtctacaattctttttctgaggtcttggctgatttcttttgattttcccatg

SEQ ID NO: 12 - DNA, 1061 bp, synthetic construct
  misc_feature 1..1061: note = Artificially synthesized
  source 1..1061: mol_type = other DNA
ctagtatttggtagcattgcctttaaattgtttaacttgggtcaaacgtttcgggtagccttccacaagcttcccacaataatttgggtgaattttggcccattcctcctgacagagctggtgtaactgagtcaggtttgtaggcctccttgctcgcacacgctttttcagttctgcccacaaattttctataggattgaggtcagggctttgtgatggccactccaataccttgactttgttgtccttaagccattttgccacaactttggaagtatgcttggggtcattgtcgtgttggaagacccatttgcgaccaagctttaacttcctgactgatgtcttgagatgttgcttcaatatatccacatactgcaccgcgtccatgatgccatctattttgtgaagtgcaccagtccctcctgcagcaaagcacccccacaacatgatgctgccacccccgtgcttcacggttgggatggtgttcttcggcttgcaagcctcccccttcttcctccaaacataacgatggtcattatggccaaacagttctatttttgtttcatcagaccagaggacatttctccaaaaagtacgatctttgtccccatgtgcagttgcaaaccgtagtctggctttcttatgtcggttttggagcagtggcttcttccttgctgagtggcctttcaggttatgtcgatataggactcgttttactgtggatatagatacttttgtacctgtttcggacacattgtgccaaagggattttaggggcgcttacctccagcatcttcacaaggtcctttgctgttgttctgggattgatttgcacttttcgcaccaaagtacgttcatctctaggagacagaacgcgtctccttccttagcgctatgacggctgcgtggtcccatggtgtttatacttgcgtactattgtttgtacagatgaacgtggtaccgccaggcgtttggaaattgctcccaaggatgaaccagacttgtggaggtctacaattctttttctgaggtcttggctgatttcttttgattttcccatg

SEQ ID NO: 13 - DNA, 169 bp, synthetic construct
  misc_feature 1..169: note = Artificially synthesized
  source 1..169: mol_type = other DNA
gtgagcagctggggctggagagacgacagggctggttgcccagggtccccaggcctctgattcctcactgattgctcttaagcaagcaggacaagaagcggtggaggagaccaagggtgcagttatgcctcagattcacttttatcacctttccttgcctctttcctag

SEQ ID NO: 14 - DNA, 135 bp, synthetic construct
  misc_feature 1..135: note = Artificially synthesized
  source 1..135: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagtt